(19) United States
(12) Patent Application Publication
(10) Pub. No.: US 2023/0227815 A1
(43) Pub. Date: Jul. 20, 2023

(54) Systems, Methods, And Compositions For A Facile Accelerated Specific Therapeutic (Fast) Pipeline

(71) Applicant: The Regents of the University of Colorado, a body corporate, Denver, CO (US)

(72) Inventors: Anushree Chatterjee, Lafayette, CO (US); Thomas Aunins, Boulder, CO (US)

(21) Appl. No.: 17/633,735

(22) PCT Filed: Aug. 10, 2020
(86) PCT No.: PCT/US20/45638

§ 371 Date: Feb. 8, 2022

Related U.S. Application Data
(60) Provisional application No. 62/884,974, filed on Aug. 9, 2019.

(51) Int. Cl. C12N 15/10; C12N 15/67; C40B 30/06; G16B 15/30; G16B 35/20

(52) U.S. Cl. CPC C12N 15/1093; C12N 15/67; C40B 30/06; G16B 15/30; G16B 35/20

(57) ABSTRACT

The present inventions describes a Facile Accelerated Specific Therapeutic (FAST) pipeline to rapidly design, built and test peptide nucleic acid treatments against mammalian or microbial genes of interest. The invention may include a bioinformatics application for facile and accelerated high throughput design of peptide nucleic acids (PNAs) that act as inhibitors of expression of specific targeted genes by binding to their mRNA to block translation, or PNA activators that can activate expression of target genes by binding to the respective promoter regions and recruitment of transcriptional activators. The invention may further involve automated and high throughput parallel synthesis of a PNA inhibitor/activator library for generation of on-site therapeutic molecules, which may reduce storage requirements, and the development of efficient delivery of therapeutic PNAs to host cells to overcome challenges of transport, toxicity, and bioavailability. The invention may further involve the testing of designed and built PNAs in a high throughput manner in a relevant infection, or mammalian cell culture model. The proposed invention may allow identification of important gene targets, and quickly generate translatable therapies that can be tested under host conditions, and most importantly develop a countermeasure platform that can be deployed on-site in the future to generate therapies in short time scales.

### CROSS-REFERENCE TO RELATED APPLICATIONS

This International PCT Application claims the benefit of and priority to U.S. Provisional Application No. 62/884,974, filed Aug. 9, 2020. The entire specification, claims, and figures of the above-referenced application are hereby incorporated, in their entirety by reference.

### GOVERNMENT INTEREST

This invention was made with government support under grant number D17AP00024 awarded by DOD/DARPA, and Grant number NNX16A069A awarded by NASA. The government has certain rights in the invention.

### TECHNICAL FIELD

The present inventions describes a Facile Accelerated Specific Therapeutic (FAST) pipeline to rapidly design, built and test therapeutic oligomers (FASTmers) against mammalian or microbial genes of interest.

### BACKGROUND

The encroaching crisis of multidrug-resistant (MDR) pathogens, incited in part by the broad-spectrum activity of many conventional antibiotics and worsened by the dwindling antibiotic pipeline, has led to an increase in research of antisense nucleic acid-based therapies. Antisense antibiotic therapies bind to bacterial nucleic acid molecules in a sequence-specific manner to inhibit the transcription and translation of important or essential genes. The sequence-specificity allows for precise targeting of a single pathogen and allows for therapies that are more adaptive to bacterial mutations than conventional antibiotics. One of the most promising of these antisense antibiotic strategies is peptide nucleic acids (PNA), synthetic peptide-nucleobase hybrids that present a cloning-free antisense strategy. Compared with other antisense strategies, PNA present better thermal stability, and better intracellular stability—PNA is not recognized by bacterial nucleases or proteases—and stronger binding due to a lack of electrostatic repulsion between PNA and nucleic acid backbones. Furthermore, PNA have demonstrated the ability to discriminate single mismatches in short sequences, allowing for a high degree of specificity. PNA have also shown to be an effective antibiotic platform.

Though PNA do offer advantages in stability and specificity, it remains necessary to both screen and experimentally test possible PNA sequences to maximize both of these qualities. However, this requires tedious design processes that involve multiple disjoint alignment programs and stability calculations. As such, there is a need to overcome the limitations in the art and develop a rapid and cost-effective process for the design, synthesis, and testing of PNA antibiotics.

### SUMMARY OF THE INVENTION

One aspect of the inventive technology includes a novel approach to counter emergent bacterial threats through the development of a Facile Accelerated Specific Therapeutic (FAST) system to design, built and test treatments within a time scale of hours to days against any emergent bacterial pathogen including multi-drug resistant (MDR) bacteria. In a preferred embodiment, the invention includes a the Facile and Accelerated high throughput design of therapeutic oligomers, referred to generally as FASTmers. In certain preferred aspects, a FASTmers may include peptide nucleic acids (PNAs) based antisense therapeutics that can block translation of a desired gene by binding to its mRNA in pathogen-specific manner for targeted inhibition of growth/killing of a desired pathogen. Additional embodiments may further include the automated and high-throughput parallel synthesis of a FASTmer therapeutic library for generation of on-site therapeutic molecules, and development of a bacterial secretion system, such as Type III secretion system, associated on a probiotic delivery system to efficiently provide therapeutic FASTmer molecules to infected host cells to overcome challenges of transport, toxicity, and bioavailability. In this preferred embodiment, the inventive technology may include the testing of one or more designed and built FASTmers in a high-throughput manner, such as in a relevant macrophage based host-infection model.

As detailed below, in one aspect of the invention, a FASTmer, or PNA Finder toolbox may provide a set of functions that allow for rapid identification and screening of potential target gene sequences for peptide nucleic acid antibiotics. The toolbox incorporates the individual tools Get Sequences and Find Off-Targets, which provide automated pipelines of the alignment tools Bowtie 2, SAMTools, and BEDTools that work together with novel Python-based tools. The Get Sequences tool may be used to identify sequences in a given bacterial genome that PNA antibiotics could potentially target, based on a user-provided list of target genes. This tool provides as output a FASTA file of these sequences, a corresponding list of the PNA peptides that may bind to each target sequence, and analysis of these peptides for possible issues related solubility and self-complementarity. In one aspect of the invention, the Get Sequences tool may automate the target gene selection process by providing users with the option to use essential gene databases to identify gene targets in a particular organism. Furthermore, transcription start site prediction may be incorporated into the sequence warnings, in order to screen PNA candidates that are targeted to non-mRNA sequences.

In one aspect of the invention, the Find Off-Targets tool may be used to identify genomic loci that a given FASTmer sequence, and preferably a PNA sequence—or set of sequences—is predicted to bind, whether or not they represent the target locus. The tool provides as an output a BED file of these alignments, a tab-delimited output file of all alignments predicted to inhibit a gene, and a count file with the sum total inhibitory alignments for each FASTmer in the given genome. Additional aspects may incorporate a method for screening FASTmer candidates based on estimates off-targeting within the commensal microbe communities of specific microbiome environments. Still further aspects may add predictions of human off-targeting.

One aspect of the current invention may include the rapid identification and synthesis of FASTmer molecules, and preferably PNA molecules designed to either increase transcription or decrease protein expression of one or more radiation responsive genes. This embodiment may use a FAST platform pipeline to design FASTmer PNA based antisense therapeutics that can block translation of any desired gene by binding to its mRNA in a gene-specific manner.

One aspect of the current invention may include the development a bioinformatics software tool configured to design a catalog of unique FASTmer molecules, and preferably PNA molecules that can target (a) essential genes in pathogenic bacteria, (b) antimicrobial resistance genes, and (c) reduce chances of off-target effect by comparison to other pathogenic bacterial strains, gut microbiome, and human transcriptome. One embodiment may adopt an automated FASTmer synthesis method and a novel micro-bot based FASTmer delivery strategy. In this embodiment, the inventive technology may include systems and methods to synthesize FASTmer s in an automated peptide synthesizer to achieve high-throughput, lower costs, and shorter time scales to generate therapy that can be used to treat patient quickly. Additional embodiments may include the use of a novel probiotic-based transport strategy to deliver the therapeutic FASTmer molecules with higher efficiency, lowered toxicity, and increased bioavailability. Additional aspects of the invention may include systems and methods for testing FASTmers designed in a high throughput host infection model. In this preferred embodiment, the present inventors may employ a high throughput screening method for the developed FASTmer molecules using: (i) standard broth cultures, and (ii) a host mimicking mammalian infection model that can provide insights about treatment performance in less than 24 hours of experiment. In one preferred embodiment, the invention may include an infection model of pathogenic S. typhimurium strain SL1344-gfp of RAW 264.7 mouse derived macrophages.

Additional aspect of the invention my include one or more of the following preferred embodiment:

1. A method for the rational design and production of therapeutic oligomers comprising:
generating a genomic library for an organism from known target genes, whole or partial genome assemblies, or biosynthetic gene clusters (BGC's) derived from microbiome gene analysis;
initiating a sequence identification function comprising the steps of:
   analyzing said genomic library and identifying a plurality of prospective gene targets whose expression may be regulated by a proposed therapeutic oligomer;
   generating a proposed therapeutic oligomer sequence corresponding to each of said prospective gene targets; and
   outputting a sequence warning for any of said proposed therapeutic oligomer sequences;
initiating an off-target sequence function to identify genomic loci that said proposed therapeutic oligomer is predicted to bind comprising the steps of:
   searching for incidental alignments between said proposed therapeutic oligomer sequence and said a genomic library;
   aligning each of said proposed therapeutic oligomer sequences to its corresponding genome assembly location and applying a user-specified number of allowed mismatches and using the proposed therapeutic oligomer sequence length parameter as the seed length;
   identifying whether one or more of said proposed therapeutic oligomer sequences overlaps with any genomic features of said genomic library;
   outputting a file identifying all potentially inhibitory alignments of said proposed therapeutic oligomer sequences; and
   outputting a file identifying all potentially off-target alignments of said proposed therapeutic oligomer sequences;
selecting one or more of said proposed therapeutic oligomer sequences wherein said selection is based on at least one of the following criteria:
   inhibition of said target gene expression;
   upregulation of said target gene expression;
   solubility of said proposed therapeutic oligomer;
   stability of said proposed therapeutic oligomer;
   presence of self-complementary subsequences in said proposed therapeutic oligomer;
   off-target alignments in coding sequences; and
   coding sequence alignments that occur near a start codon of said target gene;
synthesizing one or more of said proposed therapeutic oligomer sequences; and
testing one or more of said proposed therapeutic oligomer sequences.
   2. The method of embodiment 1, wherein said organism comprises a prokaryotic or eukaryotic organism.
   3. The method of embodiment 2, wherein said prokaryotic organism comprises a bacteria, or a multi-drug resistance (MDR) bacteria.
   4. The method of embodiment 2, wherein said eukaryotic organism comprises a mammal.
   5. The method of embodiment 2, wherein said therapeutic oligomers comprises peptide nucleic acids (PNA).
   6. The method of embodiment 5, wherein said PNA inhibits gene expression in a target host or upregulates gene expression in a target host.
   7. The method of embodiment 6, wherein said prospective gene targets comprise essential genes selected from the group consisting of: pathogenicity genes; antibiotic resistance genes; metabolism genes; radiation responsive genes; genes associated with an immune response; genes associated with a disease condition; oncogenes; anti-inflammatory genes.
   8. The method of any embodiments 5 and 6, wherein said PNA comprises a 12-mer PNA.
   9. The method of any embodiments 5-6 and 8, wherein said PNA is synthesized to include a cell-penetrating peptide.
   10. The method of embodiment 9 wherein said cell-penetrating peptide comprises (KFF)₃K.
   11. The method of any embodiments 5-6 and 8-10, wherein said PNA is synthesized using solid-state PNA synthesis using Fmoc chemistry.
   12. The method of any embodiments 1 and 11, wherein said step of synthesizing comprises the step of automated and high-throughput parallel synthesizing a library of therapeutic oligomer sequences.
   13. The method of embodiment 1, and further comprising the step of delivering said therapeutic oligomer to a target cell through a bacterial delivery system.
   14. The method of embodiment 13, wherein said bacterial delivery system comprises a bacterial delivery system having a Type III secretion system configured to deliver said therapeutic oligomer to a target cell.
   15. The method of embodiment 1, wherein said step of testing comprising the step of testing the efficacy and/or toxicity of said proposed therapeutic oligomer sequences in an in vitro or in vivo system.
   16. The method of embodiment 15, wherein said step of testing the efficacy and/or toxicity of said proposed therapeutic oligomer sequences comprises the step of testing the efficacy and/or toxicity of said proposed therapeutic oligomer sequence in a macrophage based host-infection model.
   17. A system for the rational design and production of therapeutic oligomers comprising:
a sequence identification function configured to identify gene targets from genetic databases for a target host;
a therapeutic oligomer identification and generation function comprising a target identification function configured to identify genomic loci that a therapeutic oligomer is predicted to bind, and further configured to design a plurality of unique therapeutic oligomers that exhibit at least one of the following:
   upregulate or downregulate expression of one or more gene targets in said host; and/or
   reduced chance of off-target effect by comparison to different host strains, target host microbiome, and target host transcriptomes;
an automated high-throughput therapeutic oligomer production module configured to generate said unique therapeutic oligomers;
a testing module configured to evaluate the efficacy and/or toxicity of said unique therapeutic oligomers; and
a delivery system configured to deliver said unique therapeutic oligomers to a host cell.
   18. The system of embodiment 17, wherein said host comprises a prokaryotic or eukaryotic organism.
   19. The system of embodiment 18, wherein said prokaryotic organism comprises a bacteria, or a multi-drug resistance (MDR) bacteria.
   20. The system of embodiment 18, wherein said eukaryotic organism comprises a mammal.
   21. The system of embodiment 18, wherein said therapeutic oligomers comprises peptide nucleic acids (PNA).
   22. The system of embodiment 21, wherein said PNA inhibits gene expression in a host cell or upregulates gene expression in a host cell.
   23. The system of embodiment 22, wherein said gene targets comprise essential genes selected from the group consisting of: pathogenicity genes; antibiotic resistance genes; metabolism genes; radiation responsive genes; gene associated with an immune response; genes associated with a disease condition; oncogenes, anti-inflammatory genes.
   24. The system of any embodiments 21-22, wherein said PNA comprises a 12-mer PNA.
   25. The system of any embodiments 22 and 24, wherein said PNA is synthesized to include a cell-penetrating peptide.
   26. The system of embodiment 25, wherein said cell-penetrating peptide comprises (KFF)₃K.
   27. The system of any embodiments 21-22 and 24-26, wherein said PNA is synthesized using solid-state PNA synthesis using Fmoc chemistry.
   28. The system of any embodiments 17 and 27, wherein said automated high-throughput therapeutic oligomer production module comprises a parallel automated high-throughput therapeutic oligomer production module configured to produce a library of therapeutic oligomer sequences.
   29. The system of embodiment 17, wherein said delivery system comprises a bacterial delivery system.
   30. The system of embodiment 29, wherein said bacterial delivery system comprises a bacterial delivery system having a Type III secretion system configured to deliver said therapeutic oligomer to a host cell.
   31. The system of embodiment 17, wherein said testing module comprises a testing module configured to evaluate the efficacy and/or toxicity of said unique therapeutic oligomers in an in vitro or in vivo system
   32. The system of embodiment 31, wherein said testing module configured to evaluate the efficacy and/or toxicity of said unique therapeutic oligomers comprises testing module configured to evaluate the efficacy and/or toxicity of said unique therapeutic oligomers in a macrophage based host-infection model.
   33. The system of embodiment 17, wherein said sequence identification function comprises a Get Sequence function.
   34. The system of embodiment 17, wherein said therapeutic oligomer identification and generation function comprises a Find-Off Targets function.
   35. A method of regulating gene expression in a host cell, comprising administering a therapeutically effective amount of a therapeutic oligomer produced by the method of embodiments 1-34, to a subject in need thereof.
   36. Administering a therapeutically effective amount of a therapeutic oligomer produced by the method of embodiments 1-34, to a subject in need thereof, wherein said therapeutic oligomer is co-administered with an antibiotic.
   37. Administering a therapeutically effective amount of a therapeutic oligomer produced by the method of embodiments 1-34, to a subject in need thereof, wherein said therapeutic oligomer is co-administered with an antibiotic, and re-sensitizes said subject to said antibiotic, and/or potentiate the activity of said antibiotic.
   38. A method of treating an MDR bacterial infection comprising the step of administering a therapeutically effective amount of a therapeutic oligomer produced by the method of embodiments 1-34, to a subject in need thereof.
   39. A method of treating an MDR bacterial infection comprising the step of administering a therapeutically effective amount of a therapeutic oligomer produced by the method of embodiments 1-34, to a subject in need thereof, wherein said therapeutic oligomer is co-administered with an antibiotic.
   40. A method of treating an MDR bacterial infection comprising the step of administering a therapeutically effective amount of a therapeutic oligomer produced by the method of embodiments 1-34, to a subject in need thereof, wherein said therapeutic oligomer is co-administered with an antibiotic, and potentiate the activity of said antibiotic.
   41. A method of treating an MDR bacterial infection comprising the step of administering a therapeutically effective amount of a therapeutic oligomer produced by the method of embodiments 1-34, to a subject in need thereof, wherein said therapeutic oligomer is co-administered with an antibiotic, and re-sensitizes said subject to said antibiotic.
   42. A method of regulating expression of a target gene in a subject thereof comprising the step of administering a therapeutically effective amount of a therapeutic oligomer produced by the method of embodiments 1-34, to a subject in need thereof, wherein said step of administering further comprises the step of delivering said therapeutic oligomer to a target cell through a bacterial delivery system.
   43. A method of regulating expression of a radiation responsive gene in a subject thereof comprising the step of administering a therapeutically effective amount of a therapeutic oligomer produced by the method of embodiments 1-34, to a subject in need thereof, wherein said therapeutic oligomer upregulates or down regulates expression said radiation responsive gene.
   44. A method of regulating immune response in a subject thereof comprising the step of generating a therapeutically effective amount of a therapeutic oligomer produced by the method of embodiments 1-34, and wherein said therapeutic oligomer is introduced to a bacteria, and wherein said bacteria having said therapeutic oligomers may be introduced to a target organism, and wherein said bacteria cause a phenotypic change in said target organism.
   45. The method of embodiment 44, wherein said bacteria comprises a probiotic bacteria.
   46. The method of embodiment 44, wherein said phenotypic change in said target organism comprises a phenotypic change in the target organism's microbiome.
   47. The method of embodiment 44, wherein said phenotypic change in said target organism comprises a modified immune response.
   48. The method of embodiment 47, wherein said modified immune response comprises a reduction in inflammatory cytokine expression.
   49. The method of embodiment 47, wherein said inflammatory cytokine is inflammatory cytokine IL-10.
   50. A method of regulating the microbiome of a subject thereof comprising the step of generating a therapeutically effective amount of a therapeutic oligomer produced by the method of embodiments 1-34, and wherein said therapeutic oligomer is introduced to a bacteria, and wherein said bacteria having said therapeutic oligomers may be introduced to a target organism, and wherein said bacteria cause a phenotypic change in said target organism.
   51. The method of embodiment 50, wherein said bacteria comprises a probiotic bacteria.
   52. The method of embodiment 50, wherein said phenotypic change in said target organism comprises a phenotypic change in the target organism's microbiome.
   53. The method of embodiment 50, wherein said phenotypic change in said target organism comprises a modified immune response.
   54. The method of embodiment 53, wherein said modified immune response comprises a reduction in inflammatory cytokine expression.
   55. The method of embodiment 54, wherein said inflammatory cytokine is inflammatory cytokine IL-10.
   56. A system for regulating expression of mammalian genes comprising:—
a sequence identification function configured to identify gene targets from genetic databases for a mammalian host;
a therapeutic oligomer identification and generation function comprising a target identification function configured to identify genomic loci that a therapeutic oligomer is predicted to bind, and further configured to design a plurality of unique therapeutic oligomers that exhibit at least one of the following:
   upregulate or downregulate expression of one or more gene targets in said mammalian host; and/or
   reduced chance of off-target effect by comparison to different host strains, target host microbiome, and target host transcriptomes;
a therapeutic oligomer production module configured to generate said unique therapeutic oligomers;
a testing module configured to evaluate the efficacy and/or toxicity of said unique therapeutic oligomers; and
a delivery system configured to deliver said unique therapeutic oligomers to a mammalian host.
   57. The system of embodiment 56, wherein said mammalian host comprises human.
   58. The system of embodiment 57, wherein said gene target comprises a gene target in a human that is responsive to radiation exposure.
   59. The system of embodiment 58, wherein said gene target in a human that is responsive to radiation exposure comprise the CSF2 gene, or the EPO gene.
   60. The system of embodiment 59, wherein said CSF2 gene is upregulated in response to therapeutic oligomer.
   61. The system of embodiment 59, wherein said EPO gene is downregulated in response to therapeutic oligomer.
   62. The system of embodiment 56, wherein said gene target comprise a gene selected from the group consisting of: an antibiotic resistance gene; a metabolism gene; a gene associated with a disease condition; an oncogene; an anti-inflammatory genes.
   63. The system of embodiment 56, wherein said therapeutic oligomers comprises peptide nucleic acids (PNA).
   64. The system of embodiment 63, wherein said PNA is synthesized to include a cell-penetrating peptide.
   65. The system of embodiment 64, wherein said cell-penetrating peptide comprises (KFF)₃K.
   66. The system of any embodiments 63-65, wherein said PNA is synthesized using solid-state PNA synthesis using Fmoc chemistry.
   67. The system of any embodiments 66, wherein said solid-state PNA synthesis using Fmoc chemistry comprises a parallel automated high-throughput therapeutic oligomer production module configured to produce a library of therapeutic oligomer sequences.
   68. The system of embodiment 56, wherein said delivery system comprises a bacterial delivery system.
   69. The system of embodiment 68, wherein said bacterial delivery system comprises a bacterial delivery system having a Type III secretion system configured to deliver said therapeutic oligomer to a host cell.
   70. The system of embodiment 56, wherein said testing module comprises a testing module configured to evaluate the efficacy and/or toxicity of said unique therapeutic oligomers in an in vitro or in vivo system.
   71. The system of embodiment 70, wherein said testing module configured to evaluate the efficacy and/or toxicity of said unique therapeutic oligomers comprises testing module configured to evaluate the efficacy and/or toxicity of said unique therapeutic oligomers in a macrophage based host-infection model.
   72. A system for regulating microbiome based immune regulation comprising:
generating a genomic library from biosynthetic gene clusters (BGC's) derived from microbiome gene analysis for a target organism;
a therapeutic oligomer identification and generation function comprising a target identification function configured to identify genomic loci that a therapeutic oligomer is predicted to bind, and further configured to design a plurality of unique therapeutic oligomers;
a therapeutic oligomer production module configured to synthesize said unique therapeutic oligomers, and wherein said therapeutic oligomers may be introduced to a bacteria and regulate expression of one or more genes in said bacteria or regulate one or more genes in a target organism; and
a bacterial delivery system wherein said bacteria having said therapeutic oligomers may be introduced to said target organism, and wherein said therapeutic oligomers cause a phenotypic change in said bacteria and/or said target organism.
   73. The system of embodiment 72, wherein said target organism comprises human.
   74. The system of embodiment 72, wherein said bacteria comprise a probiotic bacteria.
   74. The system of embodiment 72, wherein said therapeutic oligomer inhibits expression of polysaccharide A in said bacteria.
   76. The system of embodiment 72, wherein said therapeutic oligomer inhibits expression wcfR gene.
   77. The system of embodiment 72, wherein said phenotypic change in said target organism comprises the reduced production in inflammatory cytokines in said target organism.
   78. The system of embodiment 77, wherein said inflammatory cytokine comprises inflammatory cytokine IL-10.
   79. The system of embodiment 72, wherein said therapeutic oligomers comprises peptide nucleic acids (PNA).
   80. The system of embodiment 79, wherein said PNA is synthesized to include a cell-penetrating peptide.
   81. The system of embodiment 80, wherein said cell-penetrating peptide comprises (KFF)₃K.
   82. The system of embodiment 79, wherein said therapeutic oligomer production module comprises a therapeutic oligomer production module wherein PNA is synthesized using solid-state PNA synthesis using Fmoc chemistry.
   83. The system of embodiment 82, wherein said therapeutic oligomer production module wherein PNA is synthesized using solid-state PNA synthesis using Fmoc chemistry comprises a parallel automated high-throughput therapeutic oligomer production module configured to produce a library of therapeutic oligomer sequences.
   84. The system of embodiment 72, wherein said delivery system comprises a bacterial delivery system.
   85. The system of embodiment 88, wherein said bacterial delivery system comprises a bacterial delivery system having a Type III secretion system configured to deliver said therapeutic oligomer to a host cell.

Additional aims of the inventive technology will be evident from the detailed description and figures presented below.

### BRIEF DESCRIPTION OF DRAWINGS

Aspects, features, and advantages of the present disclosure will be better understood from the following detailed descriptions taken in conjunction with the accompanying figures, all of which are given by way of illustration only, and are not limiting the presently disclosed embodiments, in which:

FIG. 1. shows an exemplary workflow of the FAST pipeline. In one embodiment, the FAST pipeline is capable of producing a testable library of PNA FASTmer antibiotics in less than seven days, starting from a list of prospective gene targets and going through the design, synthesis, and testing of the PNA. Results of experimental testing are fed back into the pipeline to improve predictive ability and inform gene target selection.

FIG. 2. PNA Finder workflow schematic. The Get Sequences tool and Find Off-Targets tool within PNA Finder work together to identify and screen PNA based on a multi-step toolbox workflow.

FIG. 3A-B. (A-B) PNA Finder toolbox GUI. The PNA Finder toolbox runs through a graphical user interface created using the Python 2.7 module Tkinter version 8.5.

FIG. 4. shows single variable predictive efficacy with STRING interactions. Normalized growth of MDR strains was fit to a single variable regression against protein interactions, as measured by STRING database network node degree. The STRING regression represents early development of a predictive efficacy tool that may be used to better select PNA for testing.

FIG. 5. Inhibition of growth of clinical strains of ESBL K. pnuemoniae, CRE E. Coli and MDR S. typhimurium when exposed to 10 μM of respective individual PNA (shown on bars). Significance from no treatment condition is represented by an asterisk (*) when p-val <0.05.

FIG. 6. Exemplary design of FASTmer PNA inhibitor molecules.

FIG. 7. Exemplary design of FASTmer PNA activator molecules.

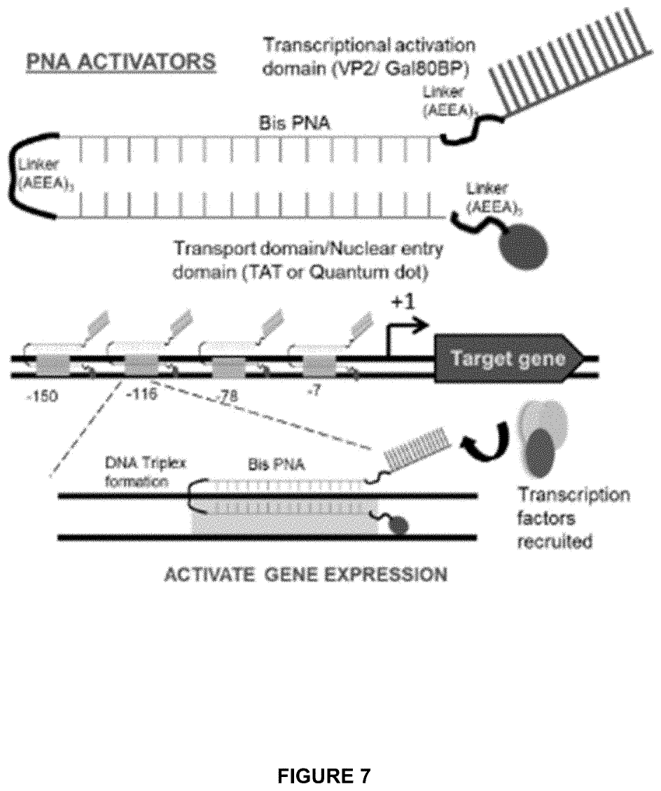

FIG. 8A-C. (A) Design of PNAs against TEM-1 beta lactamase (bla) mRNA. (B) Adding PNA restores sensitivity of drug resistant E. Coli to ampicillin. (C) Inhibition of growth of clinical strains of ESBL K. pnuemoniae, MDR E. Coli and MDR S. typhimurium when exposed to 10 μM of respective individual PNA (shown on bars). *Significance from no treatment condition (p-val <0.05).

FIG. 9. Schematic representation of synthesis of PNA sequence shown. 1,2,3,4,5,6 are Fmoc-A(Bhoc)-OH (N—((N6-(Benzhydryloxycarbonyl)aden-9-yl)acetyl)-N-(2-Fmoc-aminoethyl)glycine), Fmoc-C(Bhoc)-OH (N—((N4-(Benzhydryloxycarbonyl)cytosin-1-yl)acetyl)-N-(2-Fmoc-aminoethyl)glycine), Fmoc-G(Bhoc)-OH (N—((N2-(Benzhydryloxycarbonyl)guan-9-yl)acetyl)-N-(2-Fmoc-aminoethyl)glycine), Fmoc-T(Bhoc)-OH (N-(Thymin-1-ylacetyl)-N-(2-Fmoc-aminoethyl)glycine), HATU (O-(7-Azabenzotriazol-1-yl)-N,N,N′,N′-tetramethyluronium hexafluorophosphate) and DIEA (N,N-Diisopropylethylamine) respectively. Washing and deprotection steps can be included in the programming method.

FIG. 10 Exemplary design of a synthetic T3SS for efficient delivery of PNA-CPP to target host cells.

FIG. 11A-B. High throughput screening of PNA-CPP therapeutics in a relevant host infection model. (B) Preliminary data showing that PNA that is imported with Salmonella is able to clear infection.

FIG. 12. Schematic for an exemplary Facile Accelerated Specific Therapeutic (FAST) platform for targeting genes in prokaryotic and eukaryotic systems.

FIG. 13A-B. (A-B) Exemplary schematics for a FAST platform that may be used to develop new antimicrobial PNAs against Gram-negative Enterobacteriaceae in one embodiment thereof.

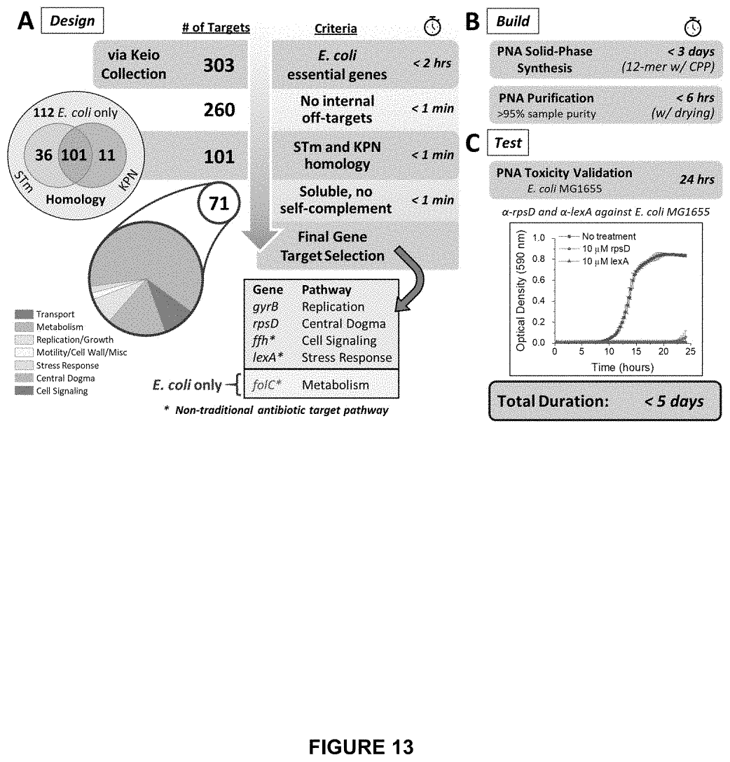

FIG. 14. Schematic flow-chart of an example of application of FAST platform in microbial engineering # (e.g. Antimicrobials, bioenergy, metabolic engineering, genome engineering).

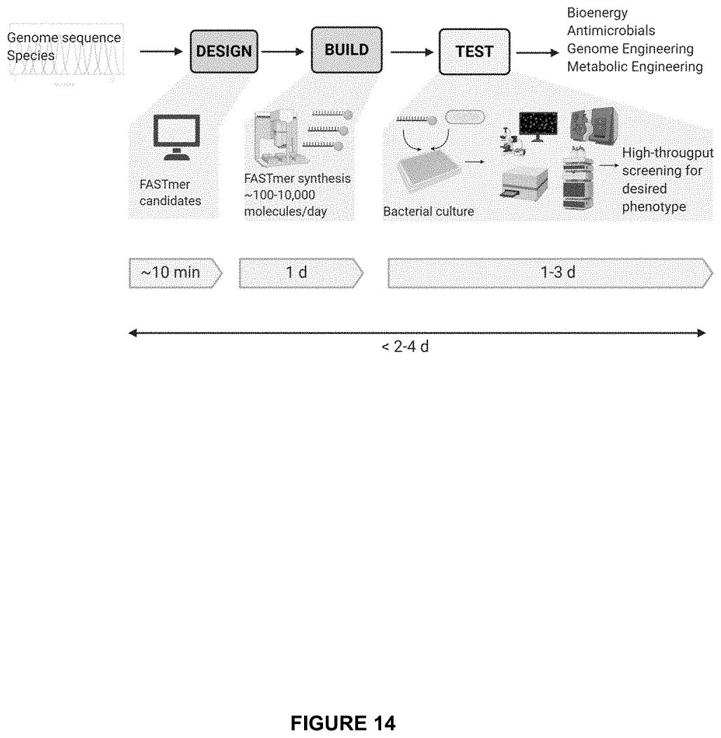

FIG. 15A-F. Exemplary bioinformatics design of FASTmers used for synthesis, purification, and monotherapy testing. (A) Bioinformatic toolbox predicts FASTmers that can target essential and non-essential genes in one or more Enterobacteriaceae including E. Coli, K. pneumoniae (KPN), and S. enterica (STm). Here, FASTmers targeting E. Coli's genome are identified and screened for internal off-targets. Candidates without off-targets are narrowed to those with homology among KPN and STm. Of the final 71 essential gene candidates and 243 non-essential gene candidates that met the thermodynamic requirements for experimental conditions, five and four FASTmers targeting essential and non-essential gene respectively were randomly chosen for assessment. Most of the FASTmers have homology to all three Enterobacteriaceae in this study, except α-folC, α-csgD, and α-fnr which are designed to be specific to E. Coli, and α-recA which is specific to E. Coli and STm. FASTmers α-folC, α-ffh, α-lexA, α-acrA, α-recA, α-csgD, and α-fnr target novel pathways of metabolism, signal recognition, stress response, transport, stress response, biofilm formation, and metabolism, respectively. FASTmers α-gyrB and α-rpsD target novel genes in traditional antibiotic pathways. (B) Following FASTmer solid-phase synthesis, the product is purified using HPLC (representative chromatogram shown), verified by LCMS (representative spectra shown), and tested against a lab strain of E. Coli (MG1655, growth curves for α-lexA and α-rpsD shown). (C) Antibiotic resistance characterization of clinical isolates of CRE E. Coli, MDR E. Coli, ESBL KPN, NDM-1 KPN, and MDR S. Typhimurium. (Left) Antibiotic resistance characterization of clinical isolates used in this study. Letters “R”, “S”, and “I” indicate drug-resistance, sensitivity, and intermediate resistance, respectively. Nine antibiotics of varied mechanisms and classes were tested including penicillins (ampicillin, AMP), cephalosporins (ceftriaxone, FRX), carbapenems (meropenem, MER), aminoglycosides (gentamicin, GEN and kanamycin, KAN), tetracyclines (tetracycline, TET), fluoroquinolones (ciprofloxacin, CIP), quinolones (nalidixic acid, NXA), and phenicols. (D) Clinical isolate monotherapy testing is done by monitoring growth at OD600 nm over 16 hours. Shown here are representative growth curves from four of the clinical isolates: NDM-1 KPN and a FASTmer control, α-nonsense, MDR E. Coli and α-ffh, CRE E. Coli and α-rpsD, and MDR STm and α-acrA. (E-F) Normalized growth (ratio of optical density of treatment to no treatment at 16 hours) of clinical isolates in the presence of treatment with 10 μM of the indicated FASTmer. FASTmers targeting essential and non-essential genes and showing at least 50% growth inhibition are shown in panels E and F respectively with significance (represented by an asterisk, p-val<0.05) determined relative to control nonsense FASTmer and no treatment, respectively. All data shown are the average of 3 biological replicates with standard deviation shown as error bars.

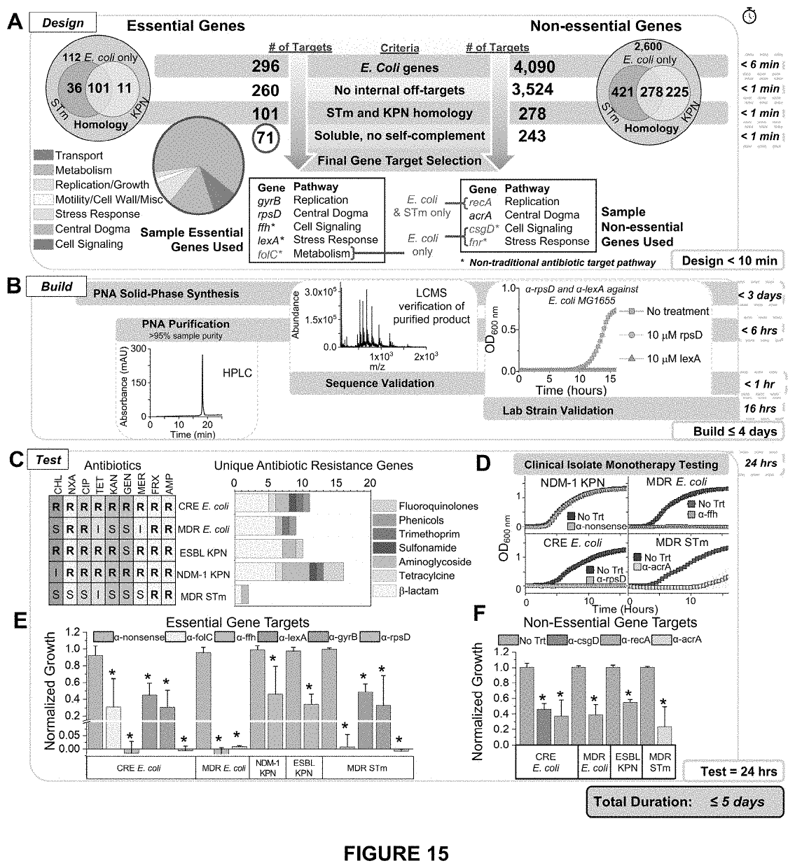

FIG. 16 Exemplary FASTmer finder process using transcriptomics-based design. Resistance profile: Minimum inhibitory concentration (MIC) of E. Coli CUS2B for 18 antibiotics. The concentrations are shown normalized to the CLSI resistance breakpoint for each of three replicates. Values ≥1 indicate resistance, while values <1 indicate intermediate resistance or susceptibility. The difference in susceptibility between ertapenem and meropenem was assessed for FASTmer targeting via RNA-sequencing.

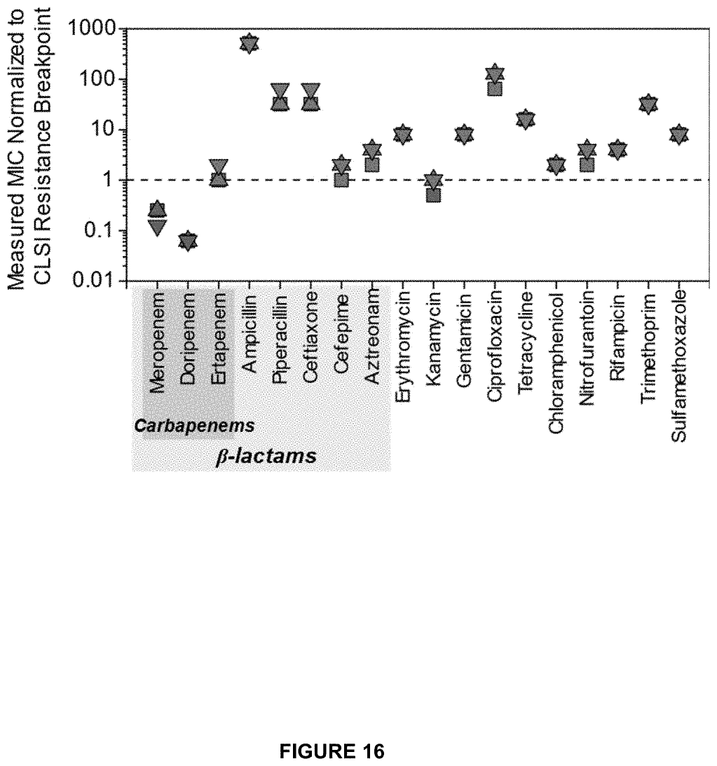

FIGS. 17A-C. Resistance profile and resistance genes of E. Coli CUS2B. (A) Minimum inhibitory concentration (MIC) of E. Coli CUS2B for 18 antibiotics. The concentrations are shown normalized to the CLSI resistance breakpoint for each of three replicates. Values ≥1 indicate resistance, while values <1 indicate intermediate resistance or susceptibility. (B) Structure of meropenem and ertapenem. (C) Antibiotic resistance genes identified in CU2SB from whole-genome sequencing data. Solid lines: antibiotics to which the gene product confers resistance. Dotted lines: antibiotics to which the gene product binds but has not been shown to hydrolyze.

FIG. 18. Example of application of FAST: Identifying genes important for drug-resistance. Exponential phase cells were grown with no antibiotic (NT), meropenem (MER), or ertapenem (ERT) and collected after 0 (NT only), 30, and 60 minutes of growth in either CAMHB with no antibiotic (NT), meropenem (MER), or ertapenem (ERT). Two biological replicates were sampled for each condition. Genes that were consistently differentially expressed in multiple conditions were chosen for FASTmer knockdown.

FIG. 19. FASTmer Finder Design, Synthesis, and Testing. Genes that were consistently differentially expressed were chosen RNA-seq data to be targeted by FASTmer. Depending on the expression profile of the genes during antibiotic challenge, different hypotheses were developed for how their knockdown would interact with each carbapenem.

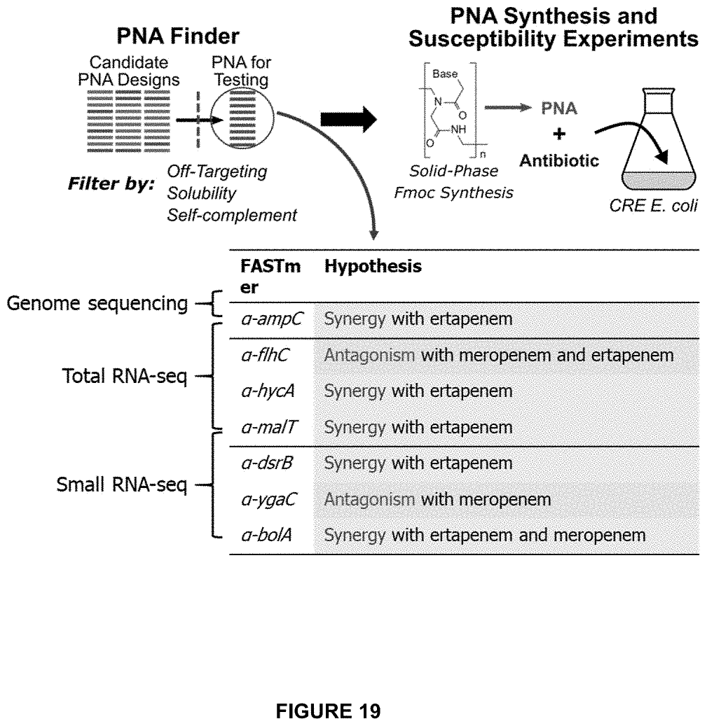

FIG. 20A-C. Short RNA-sequencing identifies regulator genes and new targets for FASTmer antibiotics. (A) Experimental setup was consistent between total RNA and short RNA sampling. The Venn diagram shows the degree to which significantly differentially expressed (DE) genes (expression levels compared to the no treatment condition at the given timepoint) overlapped across the four different conditions. Smaller pop-outs are shown below to indicate overlaps on the main diagram diagonals. (B) Detail on the 22 RNAs that were DE in at least two conditions. Log₂ (fold change) values here are with respect to the no treatment condition from the same timepoint. Bold italicized text indicates significant DE (q<0.05) vs the no treatment condition at the corresponding timepoint. (C) Time course of gene expression for four short RNA transcripts of interest. All conditions are normalized to the 0-minute timepoint expression levels, measured in duplicate just before antibiotic treatments were introduced. Asterisks indicate significant DE (q<0.05) NT=no treatment, ERT=ertapenem, MER=meropenem.

FIG. 21A-F. Application of the FAST platform to the gene targets identified in transcriptomic analysis. (A) Schematic of the FAST platform's Design, Build, and Test modules, as they were applied to the conclusions generated by our gene expression experiments. (B-F) Growth curves and endpoint cell viability assays for CRE E. Coli FASTmer-antibiotic combinations, measured in triplicate. Curves are shown for experiments in which significant interaction was observed (two-way ANOVA, P<0.05): (B) α-hycA (10 μM) combined with ertapenem (1 μg/mL); (C) α-dsrB (10 μM) combined with ertapenem (1 μg/mL); (D) α-bolA (10 μM) combined with ertapenem (1 μg/mL), and α-bolA (15 μM) combined with meropenem (0.1 μg/mL); (E) α-flhC (10 μM) combined with meropenem (0.25 μg/mL); (F) α-ygaC (10 μM) combined with meropenem (0.25 μg/mL). ERT=ertapenem, MER=meropenem. *: ANOVA interaction (antibiotic and FASTmer conditions) P<0.05, corrected for multiple hypothesis testing via the Benjamini-Hochberg procedure.

FIG. 22. Growth curves for CRE E. Coli scrambled FASTmer (scr-gene)-antibiotic combinations, measured in triplicate. Curves are shown for scrambled FASTmer for treatments in which significant interaction was observed with the anti-gene FASTmer.

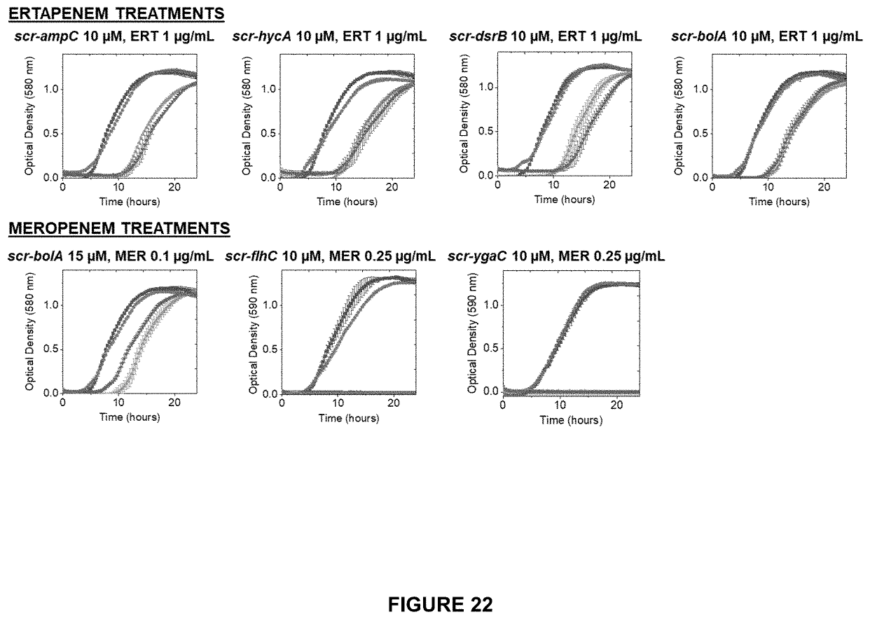

FIG. 23. FASTmer can regulate translation of targeted genes. GFP fluorescence assay for FASTmer efficacy. A GFP-expressing strain of E. Coli was diluted 1:10,000 from overnight and grown for 24-hours with IPTG at 0.01 mM to induce expression, as well as with one of three FASTmer at 10 μM: α-GFP (the anti-gene treatment), or a scrambled sequence or two-mismatch control (scr-GFP and 2MM-GFP, respectively). The two control sequence treatments did not differ significantly (P >0.1) from the positive control. *: P<0.01.

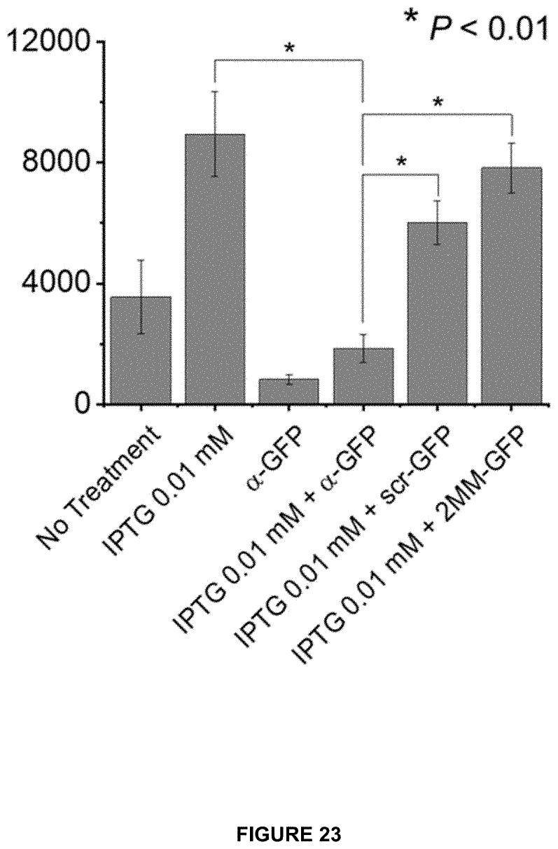

FIG. 24A-M. FAST platform generates FASTmers that can potentiate activity of traditional small-molecule antibiotics. (A) Using the STRING database of protein network interactions, we found a positive correlation, Pearson's r=0.4793 and p<0.001, between growth inhibition of FASTmer homologous with target bacteria and average node degree, the number of interactions of protein has in the average network. (B-M) Subsequent growth curves and bar plots show bacteria without treatment, or treatment with FASTmer alone (10 μM), or antibiotic alone, or FASTmer and antibiotic combined. Antibiotic concentrations were (from top to bottom) 2 μg/mL tetracycline (TET), 4 μg/mL gentamicin (GEN), and 8 μg/mL chloramphenicol (CHL). (B,F,J) The first column shows representative growth curves over 24 hours of each treatment. Panels B-E and F-G show treatment of CRE E. Coli with FASTmers targeting essential and non-essential genes, respectively. Panels J-M shows treatment of ESBL KPN with FASTmers targeting essential genes. All bar plots are the average OD (600 nm) of each treatment at 24 hours normalized to no treatment at 24 hours. S values above the bar plots were obtained using the Bliss Independence model and indicate synergistic interaction between FASTmer and antibiotic, an Asterix indicates significance at α=0.05. (N) Plotting the S values representing synergy against the target protein's average node degree shows a negative correlation (Pearson's r=−0.3017, p=0.0314). All data shown are the average of 3 biological replicates with standard deviation shown as error bars.

FIG. 25. Schematic of exemplary application of FAST platform in immune regulation and radiation countermeasures.

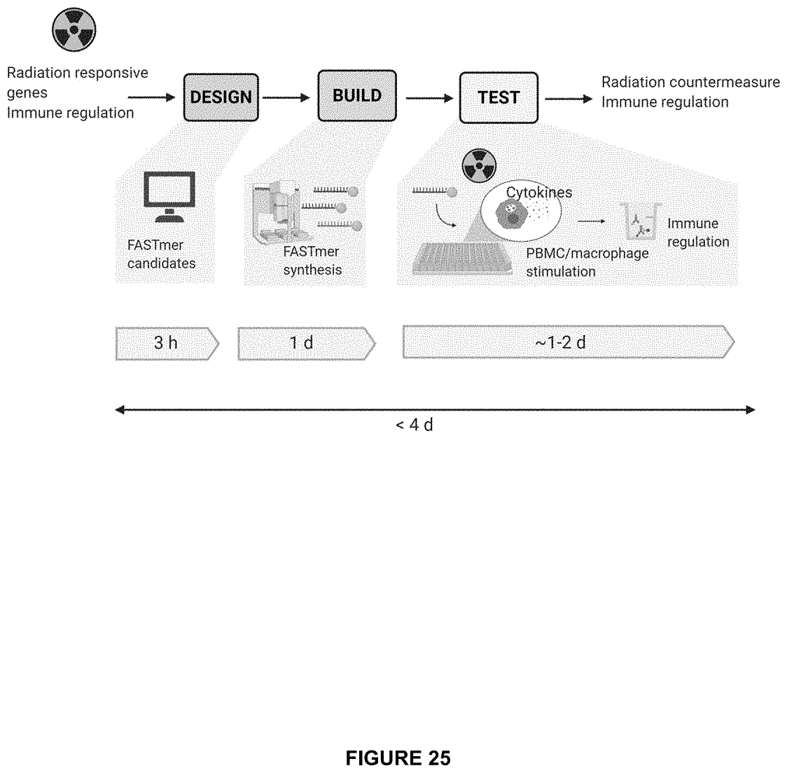

FIG. 26A-D. Use of FAST platform to regulate expression of mammalian genes. FAST platform can be used to regulate expression of mammalian genes. A,C. Cytoplasmic (A) and nuclear uptake of FASTmers (C) indicated by fluorescent dots (here FASTmers are tagged to fluorescent Quantum dots). Red color indicates mRuby nuclear stain. B,D. Example of FASTmers downregulating expression of CSF2 and EPO genes by targeting mRNA (B). FASTmers are also capable of upregulating expression of CSF2, and down-regulating expression of EPO when targeting upstream promoter regions of both genes.

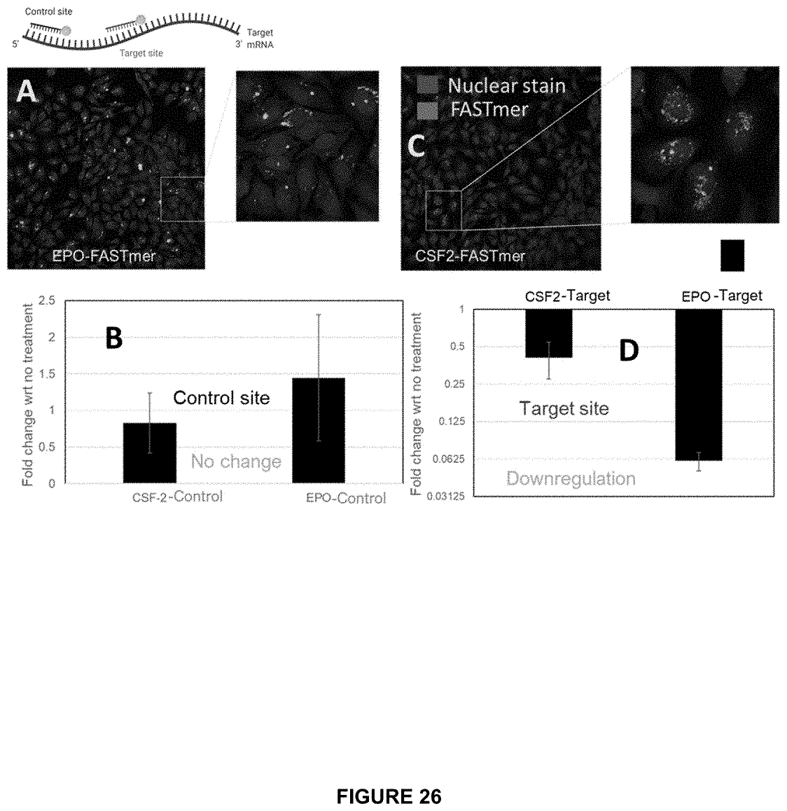

FIG. 27. Use of FAST platform to regulate expression of mammalian genes. (A) Uptake of FASTmer molecules into human cells (HeLa cells). (B) FASTmers are capable of upregulating expression of CSF2, and down-regulating expression of EPO when targeting upstream promoter regions of both genes.

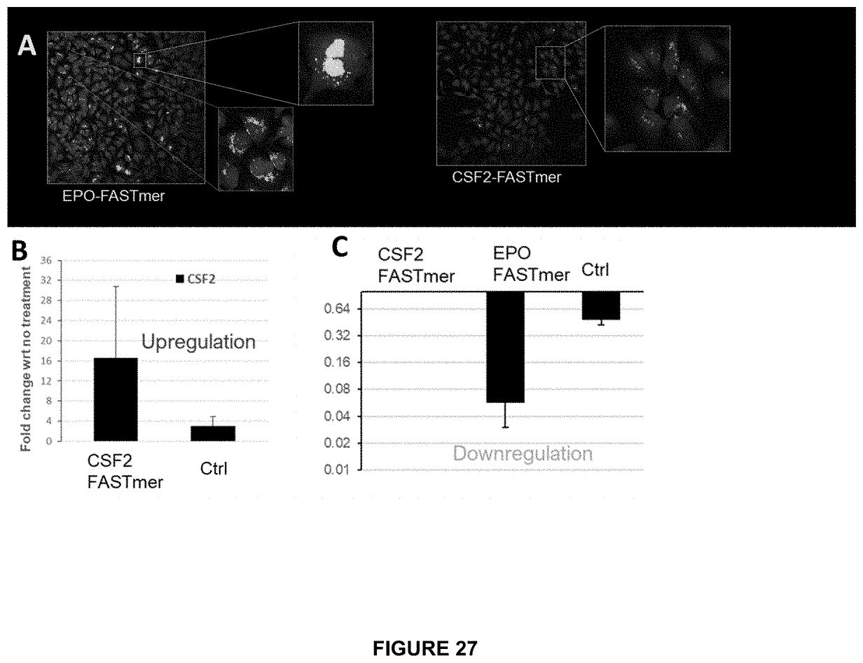

FIG. 28. Schematic diagram of exemplary application of FAST platform in microbiome and immune regulation

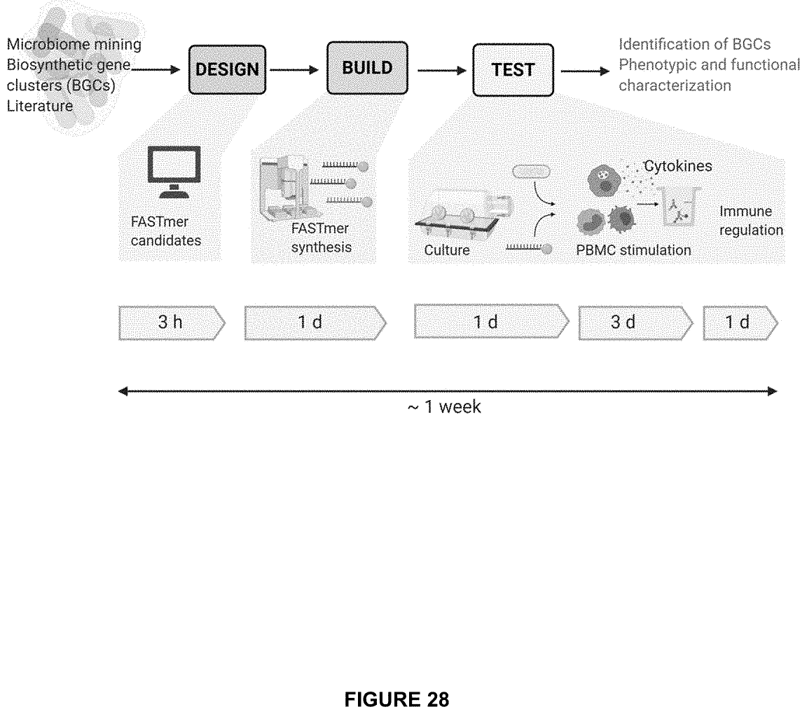

FIG. 29. FAST platform successfully reduced production of anti-inflammatory cytokine, IL-10, with lowest levels of error compared to other techniques shown in literature. FASTmers were used to inhibit expression of polysaccharide A in bacteria Bacteroides fragilis by targeting wcfR gene. Lysate from treated bacteria when exposed to peripheral blood mononuclear cell (PBMC) reduced production of IL-10 cytokine significantly compared to the wildtype (WT). Such decrease was comparable to gene knockout (APSA).

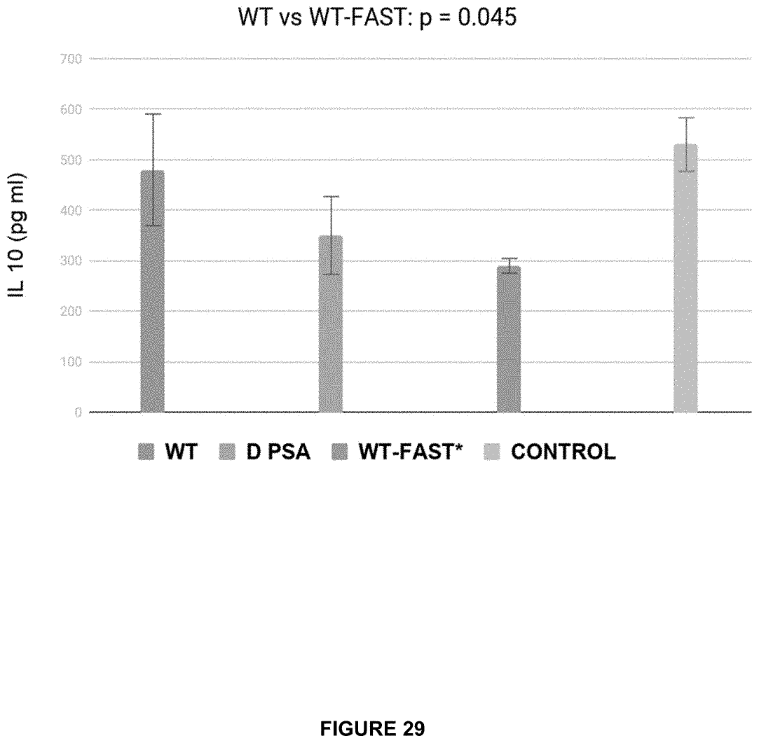

### DETAILED DESCRIPTION OF INVENTION

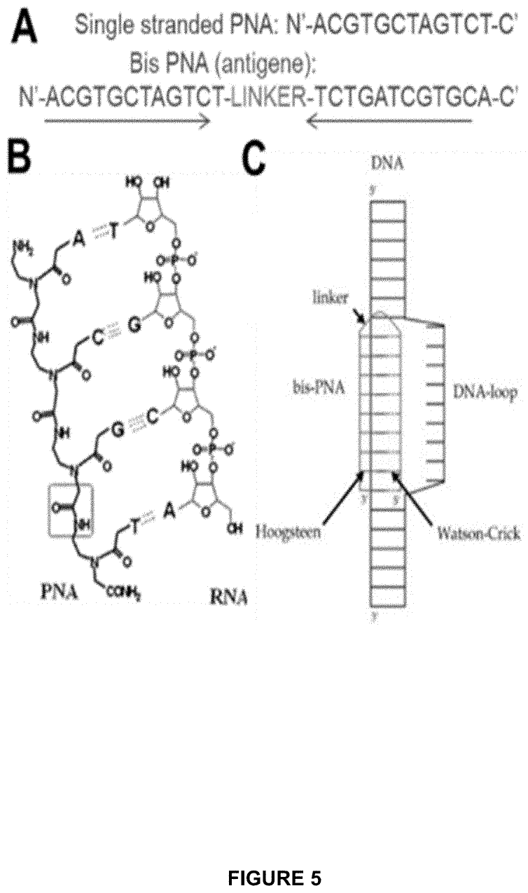

In one preferred embodiment, the invention include system, methods, and compositions for the rational design of sequence-specific therapies that target specific genes can accelerate development of novel therapeutics. FASTmers, and preferably PNA, offer a promising class of nucleic-acid targeting reagents, which demonstrate strong hybridization and specificity to their targets compared to naturally occurring RNA or DNA. PNAs are synthetic DNA analogs in which the phosphodiester bond is replaced with 2-N-aminoethylglycine units (FIG. 5B). Additionally, PNAs exhibit no known enzymatic cleavage leading to increased stability in in human blood serum and mammalian cellular extracts. This feature makes PNA especially attractive candidates for developing “cloning-free” nucleic acid therapies. As shown in FIG. 5A, antisense single stranded PNAs can be designed to bind to mRNA, whereas antigene bis-PNA oligomers have the ability to bind to double stranded DNA. Bis-PNAs are able to invade template DNA by forming “triplex invasion” complexes (FIG. 5C), which includes two complimentary homopyrimidine PNA strands connected to each other, such that one of the PNA strands is used to target a homopurine DNA binding site through combined Watson-Crick, whereas the other PNA strand interacts with the DNA strand using Hoogsteen base pairing via formation of a very stable PNA2-DNA triplex. In this invention, the present inventors may utilize use antisense PNAs for targeting mRNAs to prevent protein expression, and anti-gene PNAs for transcriptional activation.

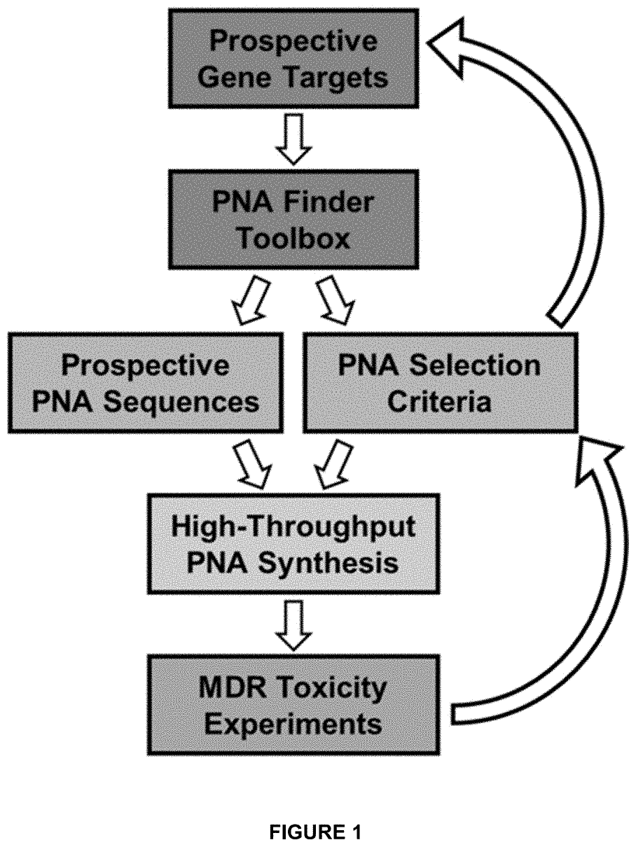

As generally shown in FIG. 1, in one preferred embodiment, the inventive FAST platform may include the generation of a list of prospective gene targets for a given target pathogen, such as an MDR bacterial species. These targets may correspond to proteins that are essential for growth, or which inhibit the MDR strain's resistance mechanism in order to re-sensitize the strain to conventional antibiotics. This list may be used as input to a PNA Finder toolbox, which provides the user with a list of candidate PNA sequences as well as several selection criteria that may be used to predict the efficacy of a given candidate. Using these data, the user filters the list of candidate PNAs and synthesizes the most promising sequences. These PNA can then quickly be subjected to efficacy testing in MDR bacterial cultures, and the resultant data can be used to both select the optimal PNA antibiotic and to inform and improve upon the PNA Finder selection process.

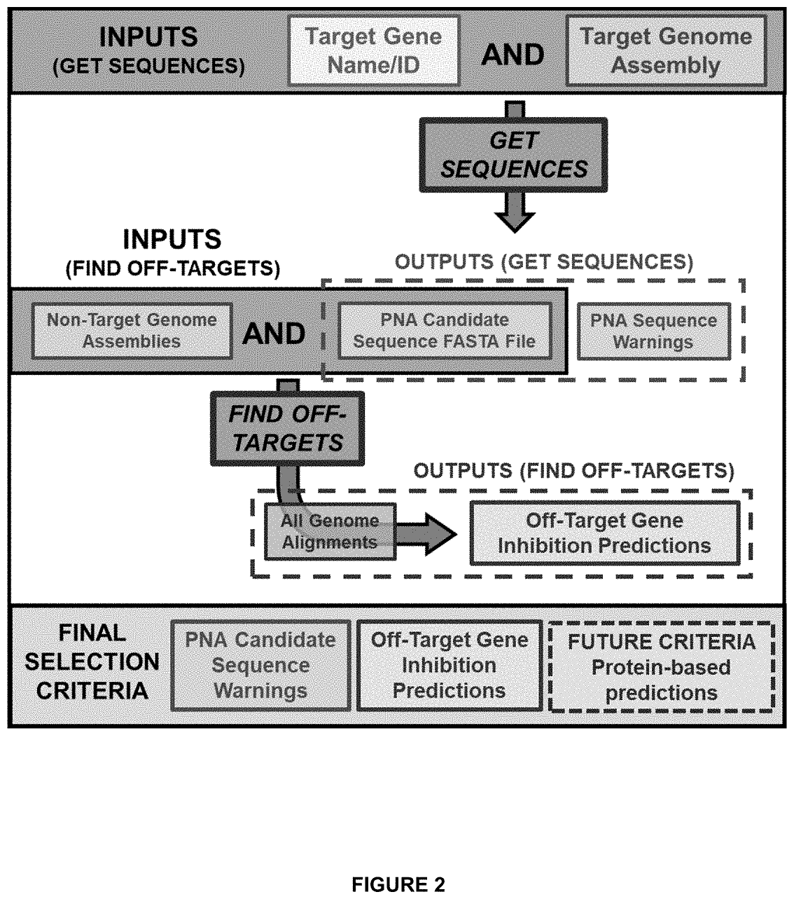

In the preferred embodiment shown in FIG. 2, the FAST platform may employ a PNA Finder, which may include a toolbox that comprises two primary functions: a “Get Sequences” tool for finding an initial list of PNA antibiotic candidates, and a “Find Off-Targets” tool for determining incidental undesired alignments of those candidates. Each of these functions may contain several sub-functions to aid in the toolbox workflow, efficiency, and the in silico PNA screening process. These tools are designed to function as a cohesive workflow, starting from a user-provided list of target genes and providing a filtered set of stable and highly specific PNA candidates that represent the most viable therapeutic options. The generalized toolbox, as described herein may also include a graphical user interface to ensure that it is a streamlined process, as well as to avoid difficulties with the command line interface on which several of its constitutive programs operate.

In this preferred embodiment, a Get Sequences tool may be used for identifying an initial list of PNA candidates and performing preliminary screening of these candidates. The tool may generate a genomic library for a host organism, which may include may take as inputs the following files: a list of gene IDs, a FASTA genome assembly for the target organism, and a corresponding GFF genome annotation file. In addition, it may incorporate the following parameters: PNA sequence length and a pair of gene coordinates relative to the +1 translation start site. The user may also be prompted to select whether they would like sequence warnings and STRING protein analysis to be included in the final output. In this embodiment, the gene IDs provided to the toolbox may represent the genes of the given organism—designated by the genome assembly and annotation files—that the user wishes to target. The Get Sequences tool reads this ID list and, for each list entry, looks through the GFF file for any coding sequence feature (designated in GFF format as “CDS”) or parent gene feature that has a matching identifier. Upon finding a matching coding sequence, the tool extracts the feature name, the start and end genomic coordinates, and the feature strand. A BED file is written with the features corresponding to each ID, and Get Sequences prints output to indicate the matches. The tool then edits the coordinates of this BED file according to the PNA sequence length and the gene coordinates parameters.

The default for PNA length is set to 12, based on competing factors. Prior research has shown a maximum of gene inhibition at PNA lengths of approximately 8 to 12 bases. This is inferred to be related to transport across the cell membrane, which worsens with increased PNA length, and binding strength and specificity, which improves with increased PNA length. However, in our PNA design we wish to minimize the expected number of off-targets in a bacterial genome, which is given by the following equation, under the simplifying assumption of total randomness of the genome (N=PNA length):

$${E}_{off - targets} = \frac{1}{{4}^{N}} \times ( genome size ) \qquad ( 1 )$$

In this example, a PNA length of 12 yields sufficient inhibition based on the prior literature and yields less than one expected off-target for even the largest bacterial genomes. Further, gene coordinates parameters may be used to designate a window near the start codon from which to propose PNA sequences. The default window is (−5 , −5) which provides a single PNA sequence that may be complementary to a region starting five bases upstream of the start codon. In one exemplary embodiment, a default 12-mer PNA would be designed to complement the base pattern *****AUG****. This default positioning may be located close to the start codon and have the highest inhibitory effect in prokaryotes. The window may be expanded according to the user's requirements, to produce multiple candidate sequences of the given length. For instance, a window of (−6, −4) with a default 12-mer PNA would produce three PNA candidates, complementary to the following base patterns: ******AUG***, *****AUG****, ****AUG*****, and the like.

In another preferred embodiment, the coordinates and strand designation of each target gene in the original BED file may be used to create a new BED file where each set of genomic coordinates corresponds to the locus that each respective PNA may target. The BEDTools function “getfasta” may then be used to produce a FASTA file of the PNA target sequences from these BED file coordinates and the input genome assembly FASTA file. An output file with the PNA sequences—reverse complements of the target sequences—may also be produced. If the options for sequence warnings and STRING database analysis are selected, these elements may be included in the output file as well. The sequence warnings function analyzes the PNA sequences for possible solubility issues, as well as self-complementary subsequences of more than six bases. The STRING database analysis may provide a network of experimentally verified, computationally predicted, and inferred protein interactions for each target gene, as well as the number of total connections between the genes of this network.

The Find Off-Targets tool may be used to search for incidental alignments between a list of PNA target sequences and a genome assembly. The tool may take as inputs the following files: a FASTA file of PNA target sequences, a FASTA genome assembly, and a corresponding GFF genome annotation file. In addition, it may incorporate the following parameters: the number of allowed alignment mismatches, PNA sequence length, and a pair of gene coordinates relative to the +1 translation start site. The user may also be prompted to select whether the tool should provide as output the total off-target counts for each PNA. The FASTA file of PNA target sequences can either be created manually by the user or taken from the output of the Get Sequences tool. The tool may use Bowtie 2 to align each PNA target sequence in the input FASTA file to the FASTA genome assembly, with the user-specified number of allowed mismatches and using the PNA length parameter as seed length. The default number of allowed mismatches may be set to zero, as many previous studies have demonstrated the high sensitivity of PNA to even a single base mismatch between the PNA and the target nucleic acid. Bowtie 2 produces a SAM alignment file as output, which is processed and indexed using the SAMTools functions “view,” “sort,” and “index.”

The resulting sorted BAM file, produced via SAMTools processing, may be used as input for the BEDTools “window” function. This function is used to identify whether a particular PNA-genome alignment in the BAM file overlaps with any genomic features, as identified by the input GFF genome annotation file. Find Off-Targets may then examine the BED file output of the “window” function to determine which PNA are expected to have off-target alignments in coding sequences, as well as which of these coding sequence alignments occur near to the start codon. These alignments to the start codon region are expected to be inhibitory to gene translation, as discussed previously. The gene coordinate inputs may be used to define the region around the start codon where inhibition is expected. The default for the Find Off-Targets tool is set to (−20, 20), based on prior observations by the inventors which showed minor translation inhibition at 17 bases upstream of a beta-lactamase start codon, but no significant translation inhibition at 23 bases downstream of the same start codon. It should be noted that this parameter is expected to vary from gene to gene, and ordinary experimentation may be required to better estimate whether a given alignment locus may cause translation inhibition.

In this preferred embodiment, the Find Off-Targets tool may produce as output a BED file of all potentially inhibitory PNA alignments. Further, if the off-target counts option was selected, the tool totals the number of potentially inhibitory off-targets for each PNA and provides those sums in a separate file. Off-target predictions may be used as another means of screening PNA candidates, either to avoid targeting other genes within a target genome or to avoid targeting another organism altogether. This function is especially valuable in PNA antibiotic design, as it allows for the design of highly specific antisense PNAs that may avoid broad antibiotic action against a microbiome environment.

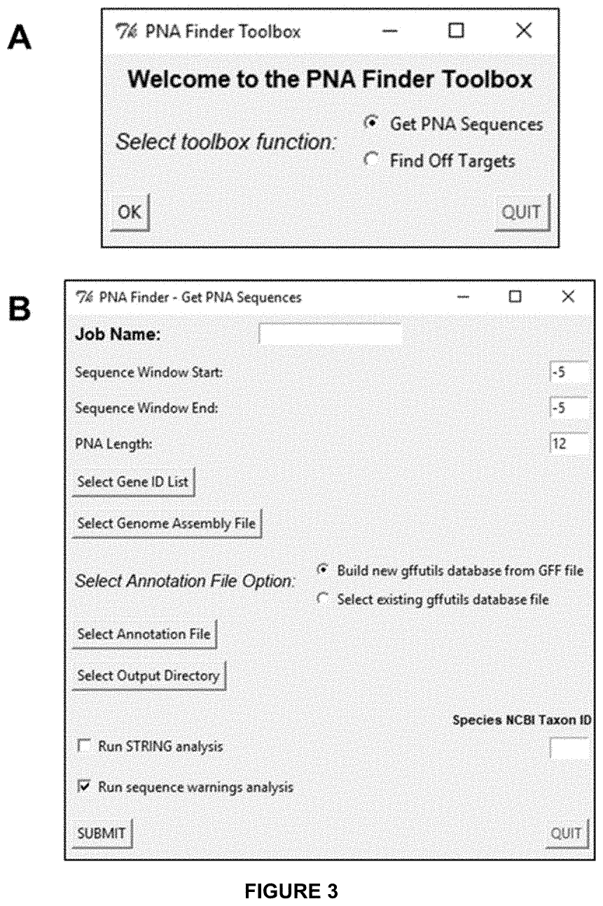

As shown in FIG. 3A, in this embodiment a UI welcome dialog may prompt the user to select either the Get Sequences or Find Off-Targets tool, and, once the selection is made, opens a dialog box to allow the user to enter the respective input parameters and input files as demonstrated in FIG. 3B. Upon completion of this dialog box, PNA Finder may check for whether all requisite inputs have been supplied and performs the toolbox function. All output files can be placed in a directory specified by the user.

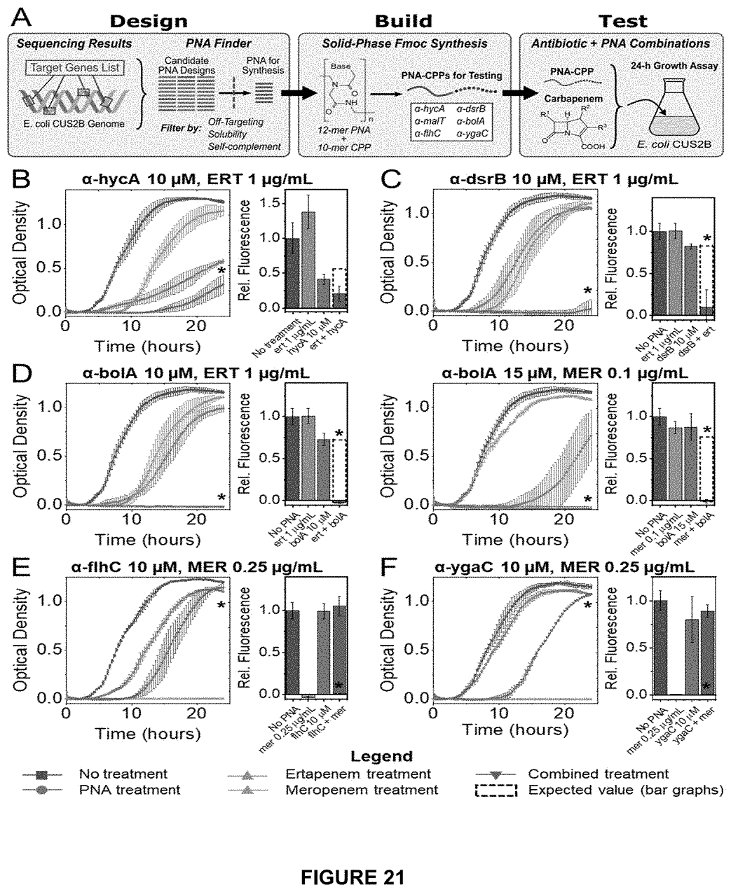

As generally described above the invention may include the design of one or more customized FASTmers that may regulate expression of a target genes in a host organism. As generally referring to FIG. 21, in one preferred embodiment, the sequences for one or more

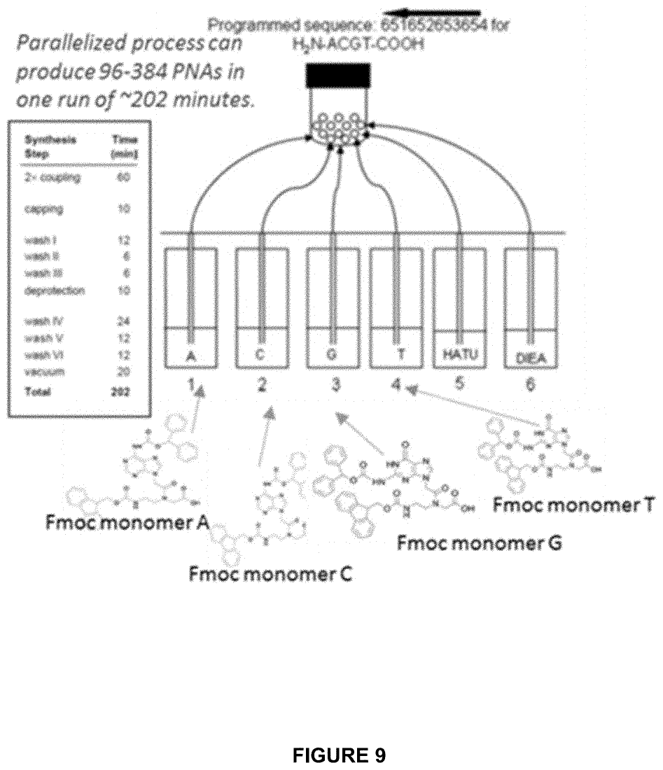

FASTmers, which may preferably be a PNA or other oligonucleotide sequence such as a dsRNA, or asRNA oligonucleotide may be rapidly produced. In this embodiment, one or more customized FASTmers, which in this embodiment are PNA, may be rapidly generated using solid-phase Fmoc synthesis as generally shown in FIG. 9.

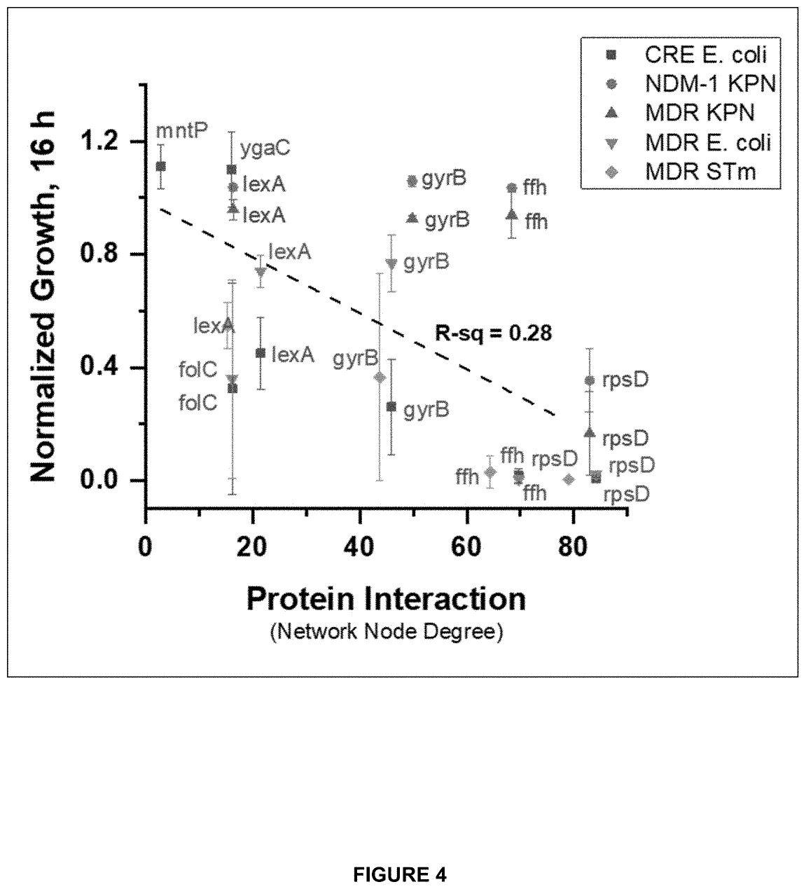

In one embodiment, an Apex 396 peptide synthesizer (AAPPTec, LLC) may be used to perform solid-state PNA synthesis using Fmoc chemistry on MBHA rink amide resin at a 10 μmol scale. Fmoc-PNA monomers were obtained from PolyOrg Inc. A, C, and G monomers are protected at amines with Bhoc groups. As shown in FIG. 4, all PNA synthesized for predictive efficacy evaluation may be synthesized with a cell-penetrating peptide, such as (KFF)₃K, which has lysine residues protected with Boc groups. PNA products may further be precipitated and purified as trifluoroacetic acid salts.

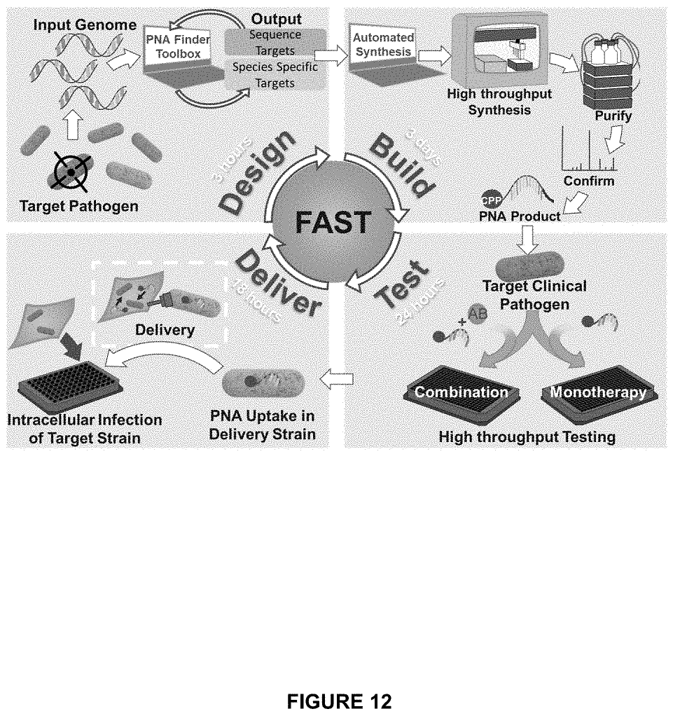

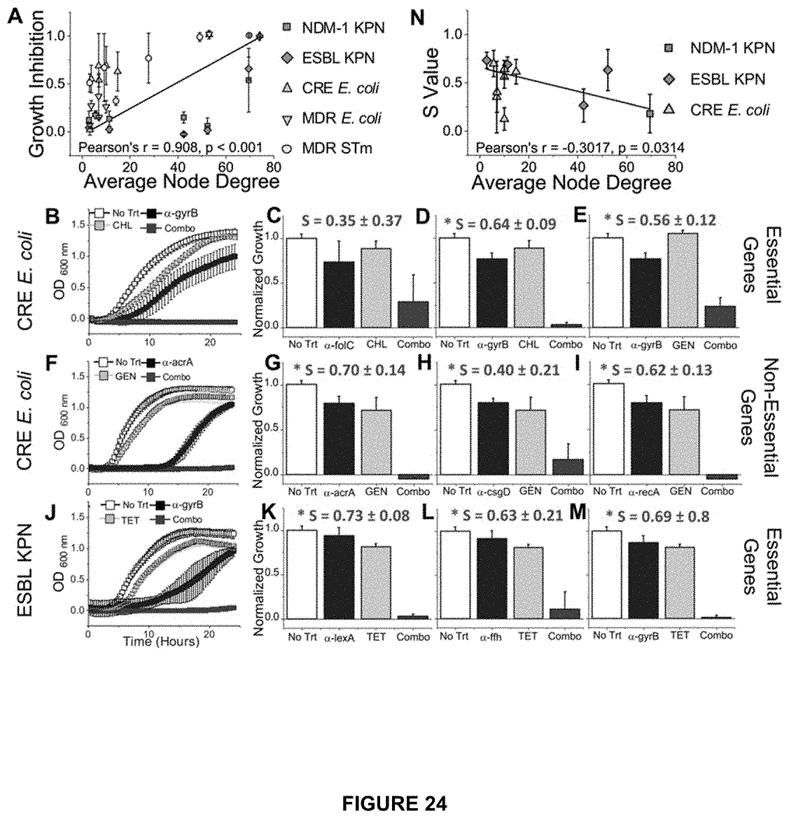

As generally described in FIGS. 12 and 21, the synthesized FASTmers may be purified and further tested in an in vitro or in vivo environment for specific activity. For example, in some embodiment the FASTmer may be evaluated. As noted above, in some instances the specific activity of a design and built FASTmer may include the up- or down-regulation of the expression of oen or more target genes, while in alternative embodiments, the FASTmer may include a specific activity of potentiating known therapeutic compounds. Fr example, as shown in FIG. 24, a FASTmer may be used to potentiate activity of traditional small-molecule antibiotics.

In one preferred embodiment, a PNA may be selected for synthesis according to application-specific needs for sequence stability and specificity, which can be ascertained from the output of the PNA Finder toolbox. In one embodiment, FAST platform synthesis process may include synthesis, purification and drying of the samples. This process may take, in some embodiment four or less days and may further achieve final sample purities of greater than 90%. In one preferred embodiment, testing of the PNA candidate efficacy in MDR bacteria occurs over the course of a 16-hour experiment, where inhibition of each antibiotic PNA is measured against that of a scrambled nonsense sequence. As shown in FIG. 1, normalized growth data is used to determine the most effective antibiotic PNA, as well as to improve the efficacy predictions of the PNA Finder toolbox. Initial work on efficacy prediction by the inventors has found a moderate correlation between STRING database protein network node degree—a measure of the connectivity of a given gene within bacterial metabolism—and normalized 16-hour growth data of MDR bacteria (R²=0.28, FIG. 4). Further collection of data, addition of variables, and development of the predictive efficacy framework may improve the predictions of PNA Finder and enhance the overall utility of the novel FAST platform system.

In one embodiment, a PNA Finder toolbox may be built using Python 2.7, as well as the alignment program Bowtie 2, the read alignment processing program SAMtools, and the feature analysis program BEDTools. Additionally, in order to run on a Windows operating system, the toolbox may incorporate the program Cygwin to provide a Unix-like environment in which Bowtie 2, SAMtools, and BEDTools can be compiled and run. The user interface for the PNA Finder Toolbox may also constructed using the Python 2.7 package Tkinter, version 8.5.

In a preferred embodiment, clinical isolates may be obtained and grown in Cation Adjusted Mueller Hinton broth (CAMHB) (Becton, Dickinson and Company 212322) at 37° C. with 225 rpm shaking or on solid CAMHB with 1.5% agar at 37° C. Clinical isolates were maintained as freezer stocks in 90% CAMHB, 10% glycerol at −80° C. Freezer stocks were streaked out onto solid CAMHB and incubated for 16 hours to produce single colonies prior to experiments. For each biological replicate, a single colony may be picked from solid media and grown for 16 hours in liquid CAMHB prior to experiments. At the start of experiment, each culture may diluted 1:10,000 in fresh CAMHB and added to either a control experiment without PNA or a 10 uM PNA condition. PNA samples were stored in 5% DMSO to aid in stability.

As used herein, PNAs may be DNA analogs in which the phosphate backbone has been replaced by (2-aminoethyl) glycine carboyl units that are linked to the nucleotide bases by the glycine amino nitrogen and methylene carbonyl linkers. The backbone is thus composed of peptide bonds linking the nucleobases. Because the PNA backbone is composed of peptide linkages, the PNA is typically referred to as having an amino-terminal and a carboxy-terminal end. However, a PNA can be also referred to as having a 5′ and a 3′ end in the conventional sense, with reference to the complementary nucleic acid sequence to which it specifically hybridizes. The sequence of a PNA molecule is described in conventional fashion as having nucleotides G, U, T, A, and C that correspond to the nucleotide sequence of the DNA molecule. Such polynucleotides can be synthesized, for example, using an automated DNA synthesizer. Typically, PNAs are synthesized using either Boc or Fmoc chemistry. PNAs and other polynucleotides can be chemically derivatized by methods known to those skilled in the art. For example, PNAs have amino and carboxy groups at the 5′ and 3′ ends, respectively, that can be further derivatized. Custom PNAs can also be synthesized and purchased commercially. Since PNA is structurally markedly different from DNA, PNA is very resistant to both proteases and nucleases, and is not recognized by the hepatic transporter(s) recognizing DNA.

As used herein, a “FASTmer,” may include an “oligomer” or “therapeutic oligomer” generated using the FAST Platform as generally described herein. In certain embodiments, a FASTmer may include or “antisense oligonucleotides,” which may include any antisense molecule that may modulate the expression of one or more genes. Examples may include antisense PNAs, antisense RNA. This term also encompasses RNA or DNA oligomers such as interfering RNA molecules, such as dsRNA, dsDNA, mRNA, siRNA, or hpRNA as well as locked nucleic acids, BNA, polypeptides and other oligomers and the like.

In yet another embodiment, the PNA comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

As used herein, the term “gene” or “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example, a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers that have been modified, whether naturally or by intervention.

As used herein, the terms “inhibit” and “inhibition” means to reduce a molecule, a reaction, an interaction, a gene, an mRNA, and/or a protein's expression, stability, function, or activity by a measurable amount, or to prevent such entirely. “Inhibitors” are compounds that, e.g., bind to, partially or totally block stimulation, decrease, prevent, delay activation, inactivate, desensitize, or down regulate a protein, a gene, and an mRNA stability, expression, function, and activity, e.g., antagonists.

The term “target sequence,” may mean a nucleotide sequence, such as a DNA sequence, or an mRNA sequence, that may be complementary to antisense molecules, and preferably an antisense peptide nucleic acid.

The term “including” is used herein to mean, and is used interchangeably with, the phrase “including but not limited to.” The term “or” is used herein to mean, and is used interchangeably with, the term “and/or,” unless context clearly indicates otherwise.

The term “therapeutically effective amount” as used herein refers to that amount of a FASTmer composition being administered which will relieve to some extent one or more of the symptoms of the disorder being treated. In reference to the treatment of a bacterial infection, and in particular a MDR infection, a therapeutically effective amount refers to that amount which has the effect of (1) reducing the infection, (2) inhibiting (that is, slowing to some extent, preferably stopping) bacterial growth, (3) inhibiting to some extent (that is, slowing to some extent, preferably stopping) bacterial pathogenicity, and/or (4) relieving to some extent (or, preferably, eliminating) one or more signs or symptoms associated with the infection.

As used herein, “subject” refers to a human or animal subject. In certain preferred embodiments, the subject is a human.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

It will be recognized by those of skill in the art that any of the DNA or mRNA sequences described above can be targeted by antisense inhibitors. Target sequences can be those of E. Coli or the homologous gene or mRNA sequence in another target bacterium. Given the benefit of this disclosure, those of skill in the art will be able to identify a target sequence and design an antisense inhibitor oligomer to target the gene or mRNA sequence. Target sites on DNA or RNA (e.g. sRNA) associated with antibiotic resistance can be any site to which binding of an antisense oligomer will inhibit the function of the DNA or RNA sequence. Inhibition can be caused by steric interference resulting from an antisense oligomer binding the DNA RNA sequence, thereby preventing proper transcription of the DNA sequence or translation of the RNA sequence.

Certain preferred embodiments provide a FAST pipeline to design and generate PNAs for treating a bacterial infection, and in particular, an MDR bacterial infection. Common drug-resistant bacteria that may include, but not be limited to: carbapenem resistant Enterobacteriaceae Klebsiella pneumonia (CREKP), MDR tuberculosis (MDRTB), MDR Salmonella enterica, MDR Salmonella typhimurium (MDRST), methicillin-resistant Staphylococcus aureus (MRSA), vancomycin-resistant S. aureus (VRSA), extended spectrum β-lactamase Klebsiella pneumoniae (ESBL K. pneumoniae), vancomycin-resistant Enterococcus (VRE), carbapenem-resistant Enterobacteriaceae Escherichia coli (CRE E. Coli), MDR Escherichia coli (MDR E. Coli), New-Delhi metallo-β-lactamase producing Klebsiella pneumoniae (NDM-1 K. pneumoniae) and MDR Acinetobacter baumannii (MRAB).

Examples of suitable probiotic microorganisms that may act as a delivery vehicle for one or more PNAs include yeasts such as Saccharomyces, Debaromyces, Candida, Pichia and Torulopsis, molds such as Aspergillus, Rhizopus, Mucor, and Penicillium and Torulopsis and bacteria such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus. Specific examples of suitable probiotic microorganisms are: Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus reuteri, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus, and Staphylococcus xylosus.

As used herein, the term “tool” means a software and/or hardware application that is designed and programed to carry out a specific task or set of tasks.

Naturally as can be appreciated, all of the steps as herein described may be accomplished in some embodiments through any appropriate machine and/or device resulting in the transformation of, for example data, data processing, data transformation, external devices, operations, and the like. It should also be noted that in some instance's software and/or software solution may be utilized to carry out the objectives of the invention and may be defined as software stored on a magnetic or optical disk or other appropriate physical computer readable media including wireless devices and/or smart phones. In alternative embodiments the software and/or data structures can be associated in combination with a computer or processor that operates on the data structure or utilizes the software. Further embodiments may include transmitting and/or loading and/or updating of the software on a computer perhaps remotely over the internet or through any other appropriate transmission machine or device, or even the executing of the software on a computer resulting in the data and/or other physical transformations as herein described.

Certain embodiments of the inventive technology may utilize a machine and/or device which may include a general purpose computer, a computer that can perform an algorithm, computer readable medium, software, computer readable medium continuing specific programming, a computer network, a server and receiver network, transmission elements, wireless devices and/or smart phones, internet transmission and receiving element; cloud-based storage and transmission systems, software updateable elements; computer routines and/or subroutines, computer readable memory, data storage elements, random access memory elements, and/or computer interface displays that may represent the data in a physically perceivable transformation such as visually displaying said processed data. In addition, as can be naturally appreciated, any of the steps as herein described may be accomplished in some embodiments through a variety of hardware applications including a keyboard, mouse, computer graphical interface, voice activation or input, server, receiver and any other appropriate hardware device known by those of ordinary skill in the art.

Any module, unit, component, server, computer, terminal, tool, engine or device exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable, and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both, which specifically includes cloud-based applications. Any such computer storage media may be part of the device or accessible or connectable thereto. Further, unless the context clearly indicates otherwise, any processor or controller set out herein may be implemented as a singular processor or as a plurality of processors. The plurality of processors may be arrayed or distributed, and any processing function referred to herein may be carried out by one or by a plurality of processors, even though a single processor may be exemplified. Any method, application or module herein described may be implemented using computer readable/executable instructions that may be stored or otherwise held by such computer readable media and executed by the one or more processors or through a cloud-based application.

The terminology used herein is for describing embodiments and is not intended to be limiting. As used herein, the singular forms “a,” “and” and “the” include plural referents, unless the content and context clearly dictate otherwise. Thus, for example, a reference to “a target gene” may include a combination of two or more such target genes. Unless defined otherwise, all scientific and technical terms are to be understood as having the same meaning as commonly used in the art to which they pertain.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Development of FAST Pipeline to Inhibit or Activate Expression of Radiation-Responsive Genes

Acute radiation syndrome (ARS) or radiation toxicity is an acute illness caused by radiation of part of or whole body by a high dose (>1 Gray or Gy) of radiation for a short period of time. Three classic radiation syndromes include hematopoietic syndrome (HS), also referred to as bone marrow syndrome, gastrointestinal syndrome (GIS), and cardiovascular (CV) or central nervous system (CNS) syndrome. Bone marrow syndrome typically occurs with a dose of 0.7 and 10 Gy. The survival rate of patient's decreases with increasing doses and primary cause of death is destruction of bone marrow which could result in infection and hemorrhage. GI syndrome typically occurs with a dose greater than 10 Gy, and survival is extremely unlikely (typically within 2 weeks) due to irreparable damage to the GI tract. CV or CNS syndrome can occur at doses greater than 50 Gy, though symptoms can occur as low as 20 Gy and considered incurable, with death occurring within 3 days due to collapse of the circulatory system.

The current radiation countermeasures are available for treatment of HS alone or in combination with GIS. HS is driven by loss of crucial growth factor-modulated hematopoietic progenitors and consequently, by large losses of circulating, functional blood cells. This condition has been shown to be treated by Colony-stimulating factors (CSFs), specifically Granulocyte-colony stimulating factor (G-CSF or CSF3) and Granulocyte-macrophage colony-stimulating factor (GM-CSF or CSF2). G-CSF is a glycoprotein produced by monocytes, fibroblasts, and endothelial cells that can induce bone marrow hematopoietic progenitors to differentiate into specific mature blood cell types released into the bloodstream. GM-CSF is a monomeric glycoprotein secreted by macrophages, T-cells, mast cells, natural killer cells, endothelial cells and fibroblasts that functions as a cytokine.

To date, there have been a number of studies involving radiation accidents where ARS patients were treated with G-CSF and GM-CSF cytokines with success. Another key protein playing a role in radiation response includes Erythropoietin (EPO), which is a glycoprotein cytokine which is secreted by the kidney in response to cellular hypoxia and stimulates red blood cell production (erythropoiesis) in the bone marrow. Erythropoietin has been shown to induce cancer cell resistance to ionizing radiation and to cisplatin, and thus can be considered a radiation countermeasure. Another family of proteins, Gamma globulin has been shown to denature after gamma radiation exposure, resulting in formation of insoluble aggregates. Gamma globulin is a protein fraction of blood plasma that responds to stimulation of antigens, such as bacteria or viruses, by forming antibodies. The catabolic rate of gamma globulin has also been shown to increase upon y-ray exposure, with increased rate of gastro-intestinal leakage, as well acute alterations of protein and gene expression.

One embodiment of the invention may include the development of a FAST pipeline to design, built and rapidly test treatments to treat radiation related pathologies. In this embodiment, rationally designed PNAs that can inhibit or activate expression of radiation-responsive genes, such as Granulocyte colony-stimulating factor (G-CSF), 2) granulocyte-macrophage-colony-stimulating factor (GM-CSF), 3) erythropoietin (EPO), and 4) gamma globulin (GG), may allow prevention or reduction of radiation-induced conditions, such as Acute Radiation Syndrome (ARS). In this embodiment, at least two types of PNAs may be generated, specifically, PNA initiators and PNA activators. PNA inhibitors may be single stranded antisense PNAs designed to bind to mRNAs of targeted gene to block translation. PNA activators may be antigene PNAs designed to bind to genomic DNA in the upstream promoter regions of targeted genes to increase gene expression.

In this preferred embodiment, the inventors may rationally design PNAs directed to gene involved in radiation responses within a subject. A PNA finder may be utilized to design a catalog of unique PNA inhibitor and activator molecules against radiation-responsive genes and reduce chances of off-target effects by comparison to gut microbiome and human transcriptome. Once target PNAs have been identified, the inventors may use a semi- or fully-automated PNA synthesis method to generate the target PNAs. In additional embodiments, a Nanoparticle-based delivery strategy may be utilized. Such a nanotechnology delivery system may provide a delivery vehicle for enhanced PNA transport, lowered toxicity, and increased bioavailability. PNAs may be introduced to mammalian cells exposed to radiation, such as gamma (γ)-radiation In this embodiment, the inventors may use a high throughput screening method for the developed PNA molecules using human macrophages, and hematopoietic stem cells exposed to γ-radiation, and in some instances microgravity to better simulate conditions in space. The target PNAs may be tested to demonstrate gene-specificity, reduced radiation response, increased transport, the lowered toxicity.

### Example 2: Design of PNA Molecules for Activation/Inhibition of Genes Involved in Radiation Response

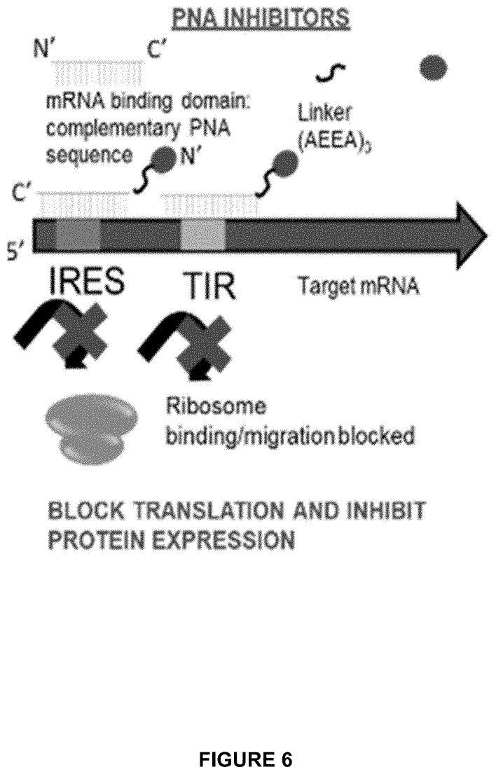

In one preferred embodiment, the invention may include the design and synthesis of approximately three to five 20-mer PNA molecules that target the translational start site (TIS) or internal ribosome entry site (IRES) of the mRNA encoded by G-CSF, GM-CSF, EPO and GG genes, with the target sequence in the middle of the oligomer, with 3-5 nucleotides flanking the target region (FIG. 6). In this embodiment, the inventors may further attach a cell penetrating peptide (CPP) or quantum dot (QD) at the N terminus of the target PNA for increased cellular entry. The inventors may further use a highly positively charged protein transduction domain of transactivator of transcription (TAT) sequence (YGRJJRRQRRR) (SEQ ID NO. 3) borrowed from HIV-1 that has been shown to successfully facilitate PNA delivery into mammalian cells and nucleus via an energy- and receptor-independent mechanism called micropinocytosis.

As shown in FIG. 5, one preferred embodiment may include the generation of novel PNA activators by using a modular approach comprising of: 1) a sequence-specific DNA-binding domain (DBD) to direct the PNA to the appropriate promoter, and 2) an amino acid sequence motif that may act as an activation domain (AD) to recruit transcription complexes to the target gene promoter region. In one example, such PNA driven activation may generate a nearly 8-fold activation of expression of gamma-globin gene using a chimeric VP2 minimal AD-PNA-TAT in mouse bone marrow cells and human primary peripheral blood cells compared to basal expression. VP2 minimal AD is a highly acidic 16 amino acid sequence (MLGDFDLDMLGDFDLD) (SEQ ID NO. 2) derived from the herpes simplex virus C terminus transactivation domain of VP16. This artificial AD has been shown to be highly effective in vitro when linked to DNA-binding domains. As such, in one preferred embodiment, the inventors may design a chimeric PNA sequence to bind to the promoter for the of G-CSF, GM-CSF, EPO and GG genes by designing 15 mer PNA centered at −150, −116, −78, and −7 positions relative to the transcriptional start sites of the gene. In order to facilitate binding to DNA, a Lysine residue may be attached to 3′ to give the PNA molecule the positive change to enhance strand invasion. For activating gene expression during PNA synthesis the inventors may further design chimeric PNA-VP2 binding domain-binding peptide chimera capable of activating transcription. Finally, for enhancing intracellular delivery, the present inventors may attach a CPP based on the TAT sequence or quantum dots as described above.

### Example 3: Developing Algorithm to Design PNA Finder Software to Rationally Designing PNAs to Activate or Inhibit Expression of Genes

The present inventors developed the first of its kind bioinformatics toolbox for designing PNAs, called PNA finder (FIG. 6). The present inventors may utilize a PNA finder software to identify sequences of target sites for the human genome using the following algorithm. The criteria for design of species specific PNAs may include: (i) Target gene is involved in radiation response, (ii) the TIR and IRES sequence is amenable to design of peptide PNAs with low melting temperature when targeting mRNA (expression inhibition), or the upstream promoter regions (−150 , −116 , −78, and −7 positions) when targeting DNA (transcriptional activation), and (iii) where possible off-target sites within the human transcriptome and between microbiome species are not present.

### Example 4: Rational Design of Peptide Nucleic Acids for Species-Centered Strategy

One preferred embodiment of the inventive technology may include the generation of novel PNA molecules configured to be directed against a gene of interest. In one preferred embodiment, PNA molecules may be designed to prevent translation of one or more essential genes within a pathogenic organism, such as MDR strains of Escherichia coli, Salmonella typhimurium, and Klebsiella pneumonia, and Methicillin Resistant Staphylococcus aureus (MRSA). In this embodiment, the present inventors may design a 12-mer PNA molecule that targets the translational start site (TIS) or ribosome binding site (RBS) of the mRNA encoded by an essential gene. Such 12-mer long PNAs may be designed against genes in pathogens using a stepwise targeting method, such that antisense oligomers are designed with the target sequence in the middle of the oligomer, with 3-5 nucleotides flanking the target region.

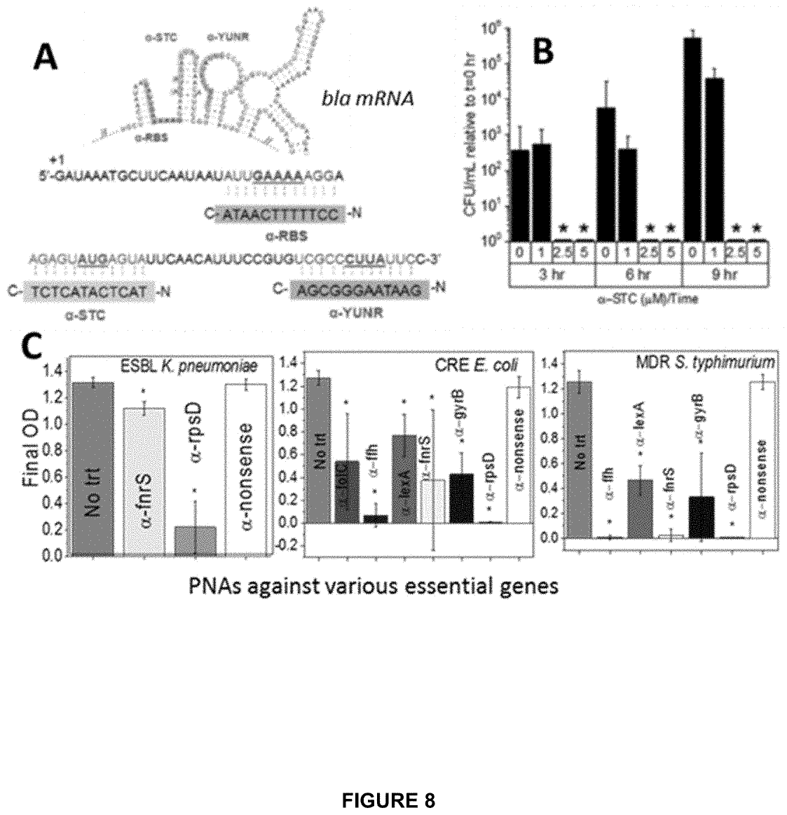

As shown in FIG. 8A, in one embodiment, the present inventors designed 12-mer PNAs oligomers, α-RBS and α-STC against the ribosome binding site (RBS) and start codon (STC) of TEM-1 β-lactamase (bla) mRNA respectively to prevent the ribosomal binding and ribosomal migration respectively, both causing inhibition of translation of bla transcript to prevent the production of active β-lactamase enzyme. The 12-mers were conjugated, to positively charged (KFF)₃K cell penetrating peptide (CPP) to increase transport across the membrane into gram-negative bacterial cells. As shown in FIG. 8B, both α-RBS and α-STC showed no off-target activity and were capable of re-sensitizing drug-resistant E. Coli to β-lactam antibiotics including restoring bactericidal activity) exhibiting 10 fold reduction in the minimum inhibitory concentration (MIC). Using the same design criteria, the present inventors further created novel PNA molecules targeting six essential genes in E. Coli including, folC involved in metabolism, ffh which is involved in cell signaling, lexA, a key regulator of stress response, and fnrS, a small Hfq binding RNA, rpsD involved in protein biosynthesis, and gyrB involved in DNA replication. These PNAs have been found to be effective in both lab and clinical strains of MDR E. Coli. Homologous PNAs were also able to target Klebsiella pneumoniae and Salmonella typhimurium a shown in FIG. 1C.

### Example 5: Developing Algorithm to Design PNA Finder Software to Rationally Designing PNAs for One or More Pathogen(s)

As noted above, in one embodiment, the inventive technology may utilize a software tool generally referred to as PNA finder that may provide sequences of target sites for a given genome using the following algorithm. The criteria for design of species specific PNAs may include: (i) target gene is essential, (ii) evidence that gene silencing of target and/or inhibition of cognate protein is growth inhibitory, (iii) the TIR and RBS sequence is amenable to design of peptide PNAs with low melting temperature, (iv) where possible off-target sites within and between species are not present in TIR and RBS sites, (v) for targeting multiple strains homologues are present in a desired number of species, and (vi) the TIR of the mRNA has at least two base pair between species when designing unique PNAs.

Additionally, the present inventors may perform bioinformatics analysis to identify the start codon sites of essential genes in a range of gram-negative and positive bacteria. The database of essential genes and NCBI BLAST may be used to identify to essential gene homologoues present in the given number of species. In one embodiment, an Artemis program may be used to extract −5 to +5 bases relative to the start codon of the TIR and RBS from the genome sequences relative to start site codon. The 10 bp of TIR and RBS from gene homologues may be aligned using Clustal X version 2.1 and number of mismatches between species may be determined. The predicted thermal stability, as determined by the meting temperature (Tm), of PNA/RNA duplexes may further be determined using mathematical formula reported in Giesen et al. Genomic analysis of possible binding sites may be conducted in Artemis using a cut-off of 2 base pair mismatches.

To examine if the number of predicted PNA sequences can be used to discriminate between closely related species, the present inventors may use a PERL script to identify the start codon positions −5 to +5 relative of each start codon of essential genes. The 10 bp may be used for an all-against-all comparison using a standalone BLAST to design PNAs that are species specific. Off-target binding affinity may be determined at three levels: (i) within the transcriptome of the organism, (ii) across bacterial species, and (ii) human transcriptome. To ensure that the chosen 12-mer does not have sequence homology to any other target inside bacteria, the 12-mer PNA may be aligned using Clustal X version 2.1 to (i) its own bacterial genome, (ii) across desired number of bacterial genomes, (iii) across human transcriptome and genome (for any potential side-effects). Genomic analysis of possible binding sites may also be conducted in Artemis using a cut-off of 2 base pair mismatches. Only PNA sequences that uniquely target bacteria of interested may be considered. For example, in one embodiment, unique PNAs will be designed against known antimicrobial gene sequences obtained from the Comprehensive Antibiotic Resistance Database (CARD).

### Example 6: Building PNAs Using a Facile Synthesis Procedure to Synthesize Peptide Nucleic Acids-Peptide Conjugates on an Automated Peptide Synthesizer

In one embodiment, the inventive technology may include an automated parallel high-throughput in-lab synthesis capable of producing a plurality of PNAs per run in a short period of time, such as less than a day, based on protocol adopted from Matysiak et al., Weiler et al., and more recently Joshi et al. In this embodiment, PNA oligomers can be synthesized using standard solid phase manual or automated peptide synthesis (such as Prelude peptide synthesizer), using either tert-butyloxycarbonyl (tBoc) or 9-fluorenylmethoxycarbonyl (Fmoc) protected PNA monomers. For example, for PNA-CPP sequence of N terminal-KFFKFFKFFK-AEEA(linker)-CACCGGCAAGTG-C terminal (SEQ ID NO. 1), firstly, the CPP peptide portion (KFF)₃K of the PNA-CPP conjugate may be synthesized on the peptide synthesizer using normal automatic mode using a Fmoc-D-Lys (Boc) Wang resin (110 mg, 0.51 mmol/g). This will be followed by PNA synthesis using Fmoc protected PNA monomers with exocyclic amino acid groups of A, T, G and C using the single-shot delivery feature of the machine.

The synthesis of PNA may be started on Fmoc-D-Lys(Boc)-Wang resin (50 mg, 0.78 mmol/g). The Fmoc protecting group can be removed by using 20% piperidine in Dimethyformamide (DMF) twice for 5 min each. This can further be followed by download of resin by partial coupling to free amino acid groups. The unreacted free amino acids may be capped by adding PNA-capping solution (2 ml, for 5 min) containing 5% DIEA. The resin may further be washed and dried. The downloading can be measured in a UV Spectrophotmeter (Nanodrop) at 290 nm. The downloaded resin may be kept in the automated synthesizer, and the coupling (0.5 ml of each PNA monomer, 0.3 ml HATU, and 0.3 ml 196.3 mM DIEA), washing (with DMF, MeOH, and DCM), deprotection, and washing steps may be repeated automatically in a continuous way until and exemplary 12-mer PNA product is obtained—although, as noted elsewhere different sized PNAs may be obtained. The final products of PNA-CPP may be purified with semi-preparative HPLC using C-18 column, and characterized using NMR and MALDI-TOF.

### Example 6: Enhancing PNA Molecule Delivery Using Bacteria Based “Micro-Robots” Using Type III Bacterial Secretion Systems

To address the issue of poor PNA transport properties, in one embodiment, the inventive technology may repurpose bacterial secretion systems, such as Type III (T3SS) or Type IV secretion systems, to deliver PNAs. Notably, T3SS are molecular machines used by many gram-negative bacterial pathogens including pathogens Shigella, Yersinia, Salmonella and Pseudomonas, to inject proteins, known as effectors, directly into eukaryotic host cells. These proteins manipulate host signal transduction pathways and cellular processes to the pathogen's advantage.

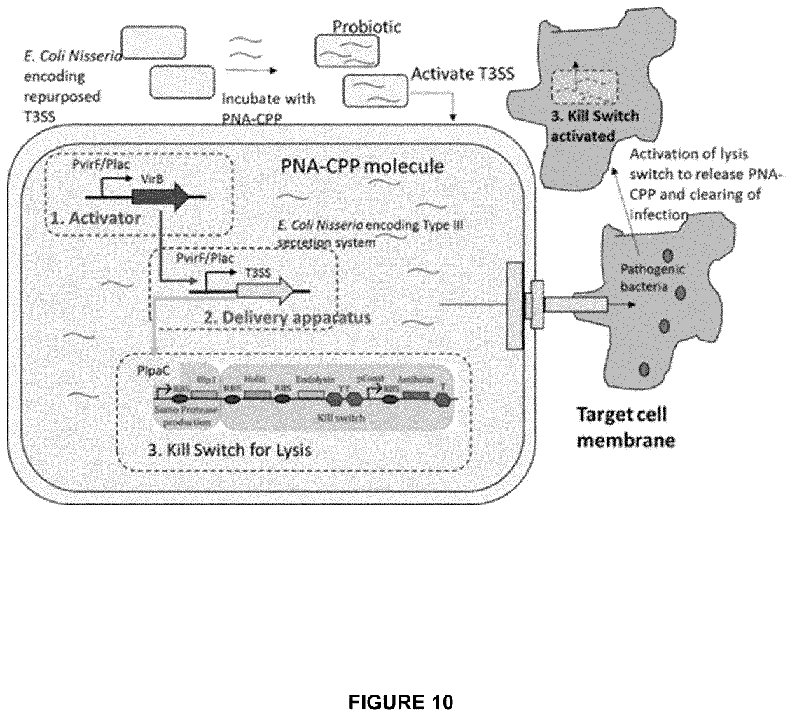

As generally shown in FIG. 10, in one preferred embodiment, the inventors may re-purpose the intrinsic Type III secretion system in a gram bacterium, such as Salmonella, may uptake and deliver PNAs to a target eukaryotic cell. In another embodiment, a T3SS function may be introduced in a non-pathogenic strains of bacteria, such as E. Coli Nissle 1917, a probiotic strain that is easily culturable, and been tested in humans for treatment of irritable bowel syndrome. In this embodiment, a T3SS from a pathogen such as Shigella flexneri may be incorporated into a synthetic biology based approach where such a protein delivery system may be composed of two parts: (i) a −31 kb long minimal DNA sequence that contains operons required for a functional T3SS from S. flexneri, and (ii) the transcriptional activator VirB to induce expression of the T3SS.

In another preferred embodiment shown in FIG. 10, the present inventors may include a kill switch circuit under the control of the Ipac promoter (Ipac is the native Shigella T3SS encoded transloacator protein that gets activated once Shigella invades a mammalian cell) to activate cell lysis once E. Coli enters mammalian cells. This may address both bio-safety concerns that E. coli Nisseria 1917 should be killed once it has entered the mammalian cell, as well as, result in efficient secretion of the PNA-CPP molecules. In this preferred embodiment, a kill switch design is based on the expression of a holin and antiholin. Holin is a protein that forms pores in cell membranes. Anti-holin forms a dimer with holin, which is not active. Once pores are formed by holin, lysozyme can access the periplasmic space and degrade the cell wall, causing cell lysis.

### Example 6: Testing the PNA Designed in a High Throughput Host Infection Model

In one embodiment, PNAs generated by the FAST system pipeline may undergo in vitro screening in broth cultures. Bacterial cultures of each individual gram negative and gram positive strain may be grown in broth or other appropriate medium. PNA molecules may be designed for each strain to either target them individually or in combinatorial manner. Scrambled PNA sequence may be used as control. PNAs would be supplied in a range of concentrations (0-50 μM) to the various combination of cultures for a period of 24 hours. The number of viable cells remaining at the end of this time point may be measured using colony forming unit analysis. The dominant strains in the culture would be identified by sampling liquid culture at end of experiment and measuring relative distribution of the strains using pathogen specific primers in a quantitative PCR assay.

### Example 7: High Throughput Fluorescent Quantification of Bacterial Load in Macrophages after PNA Treatment in a Macrophage Infection Model

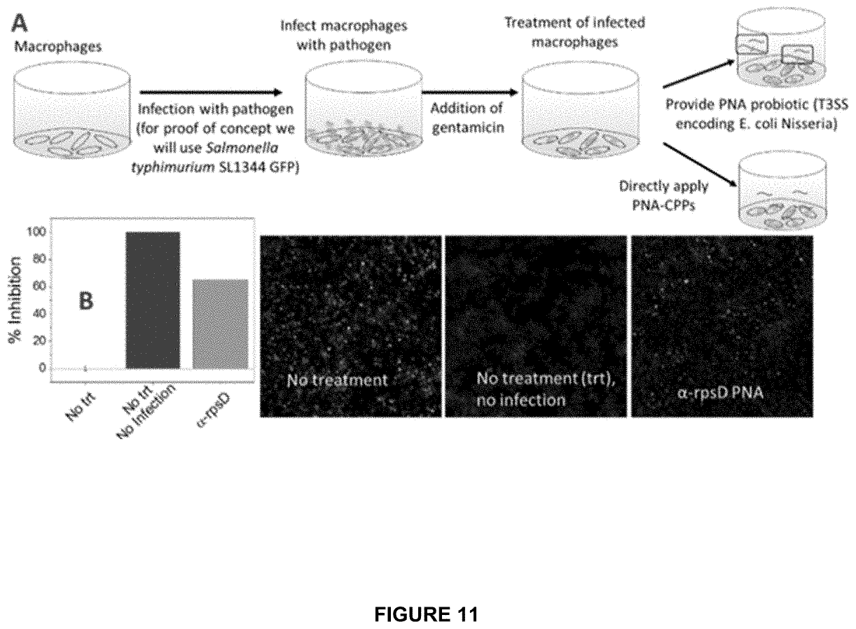

As generally shown in FIG. 11, the inventive technology may include a high-throughput fluorescent macrophage infection assays that may quantify the effectiveness of select PNAs. In one preferred embodiment, virulent S. Typhimurium in an SL1344 background may be used since it is a natural pathogen of mice and is able to infect and replicate within the host macrophages allowing the inventors to study active infection and potential clearance of that infection in vitro. Notably, Salmonella enters macrophages through either phagocytosis or through use of Type III secretion system encoded by the Salmonella pathogenicity island-1 proteins. This infection of the macrophage can lead to death of the macrophage as well as further infection of other cell types in the body and systemic infection due to proliferation of bacteria within the Salmonella-containing vacuole through expression of proteins on the Salmonella pathogenicity island-2. This strain is a BSL2 pathogen that is easy to grow and genetically tractable. It may further express GFP from the chromosome under the control of the sifB promoter, which is induced upon infection of mice and macrophages but not during growth in broth. S. Typhimurium replicates 10-15-fold within 18 hours in these cells and, as such, compounds that inhibit replication may be easily detected.

In this embodiment, the present inventors may infect at time zero with a multiplicity of infection (MOI) of 90 bacteria for each host cell, and 70% of macrophages become infected. Forty-five minutes after infection an antibiotic, such as gentamicin may be added [100 μg/mL final] to kill remaining extracellular bacteria. At 2 hours post-infection, the medium may be replaced with fresh medium containing PNAs over a dose range (0-50 μM) or DMSO (control) and gentamicin [10 μg/mL] to prevent replication of any remaining extracellular bacteria. At 18 hours post infection, cells are incubated with MitoTracker® (red, an indicator of mitochondrial membrane potential), fixed with 2.5% paraformaldehyde, and incubated with DAPI (blue). Two 10× images from each well in 3 different channels (red, blue, green) are captured on an automated microscope, the Cellomics ArrayScan VTI HCS Reader (Thermo Scientific) in the HTS facility. Quantitative image analysis may be automated using MATLAB. In this embodiment a target parameter may include the average area of GFP+ pixels, normalized to total area within the macrophage. This methodology is quantitative, unbiased, and can process samples faster than is possible manually. All images may be cataloged and stored for future reference and reanalysis as needed. 504, of supernatant may use to determine lactate dehydrogenase release as a measure of cytotoxicity using the Pierce LDH cytotoxicity assay kit.

### Example 8: Application of FAST: Identifying Genes Important for Drug-Resistance

The present inventor utilized the Facile Accelerated Specific Therapeutic (FAST) platform to create gene-specific antisense peptide nucleic acids (PNAs) molecules designed to inhibit protein translation. FAST PNAs were designed to inhibit the pathways identified in our transcriptomic analysis, and each PNA was then tested in combination with each carbapenem to assess its effect on the antibiotics' minimum inhibitory concentrations. We observed significant treatment interaction with five different PNAs across six PNA-antibiotic combinations. Inhibition of the genes hycA, dsrB, and bolA were found to re-sensitize CRE E. Coli to carbapenems, whereas inhibition of the genes flhC and ygaC was found to confer added resistance.

### Example 9: E. Coli CUS2B: A Multidrug-Resistant Enterobacteriaceae with Partial Carbapenem Resistance

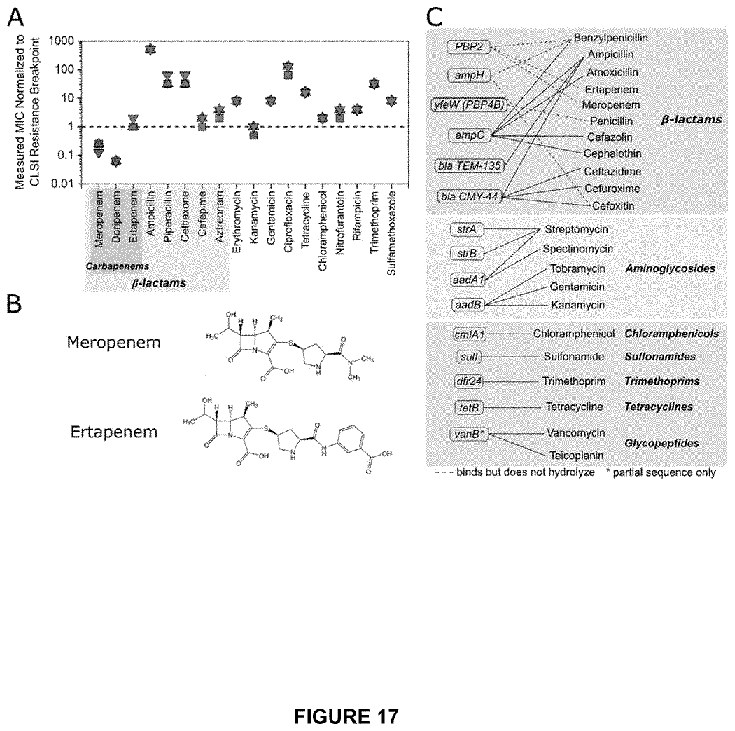

To validate the E. Coli CUS2B resistance phenotype observed in the clinic, we measured the isolate's minimum inhibitory concentrations (MIC) for a variety of antibiotics from different classes (FIG. 17A). We found that E. Coli CUS2B was resistant to almost all antibiotics (based on breakpoints defined by the Clinical & Laboratory Standards Institute), including multiple penicillins and ertapenem. Two potent carbapenem antibiotics, meropenem and doripenem, were the only drugs to which the clinical isolate was susceptible. To investigate this partial carbapenem resistance, we focused on the E. Coli CUS2B response to ertapenem and meropenem. The structures of meropenem and ertapenem differ in the pyrrolidinyl ring's position 2 side chain (FIG. 17B). Meropenem's substituent amide group is thought to be responsible for increased potency against gram-negative organisms in comparison to imipenem. At this position ertapenem has a benzoate substituent group, which imbues the molecule with a net negative charge and increases its lipophilicity, resulting in increased plasma half-life but decreased affinity for membrane porins.

### Example 10: Resistance Factor Identification Via Whole-Genome Sequencing

The present inventors performed whole genome shotgun sequencing for two purposes: (1) to create a genome assembly that could be used for antisense PNA design, and (2) to search for genomic contributions to the resistance phenotype. Using the ARG-ANNOT database, we found that the strain encodes fifteen genes related to antibiotic resistance (FIG. 17C), including six associated with β-lactams either as penicillin binding proteins (PBPs) or β-lactamase enzymes. Four of these six—labeled by ARG-ANNOT as ampC1, ampC2, ampH, and a generic PBP—are encoded chromosomally, and have high-similarity homologues in E. Coli reference strains. The generic PBP, which was found to identically match the protein sequence for E. Coli MG1655 mrdA/PBP2, is known to bind both carbapenems tested; PBP2 is the PBP to which meropenem and ertapenem demonstrate greatest activity. The genes ampC1 and ampH show >96% homology with E. Coli MG1655 PBPs yfeW/PBP4B and ampH/AmpH, respectively, and neither has been found to bind with carbapenems. The gene ampC2 shows 98% nucleotide homology with E. Coli MG1655 β-lactamase ampC/AmpC but has ten altered codons from the reference sequence. Additionally, the −35 to −10 promoter region for the CUS2B ampC has four mismatched nucleotides from the same promoter region in MG1655, which may alter its expression level relative to the basal non-resistant levels in the reference strain. The remaining two β-lactam-associated genes are plasmid encoded; these are the β-lactamases TEM-135 and CMY-44. E. Coli CUS2B also encodes the outer membrane porins OmpA, OmpC, and OmpF, the mutation or downregulation of which may influence carbapenem efficacy. These three proteins have, respectively, 95%, 90%, and 90% nucleotide homology with the corresponding genes in E. Coli MG1655.

### Example 11: Profiling Gene Expression in Response to Ertapenem and Meropenem Treatment

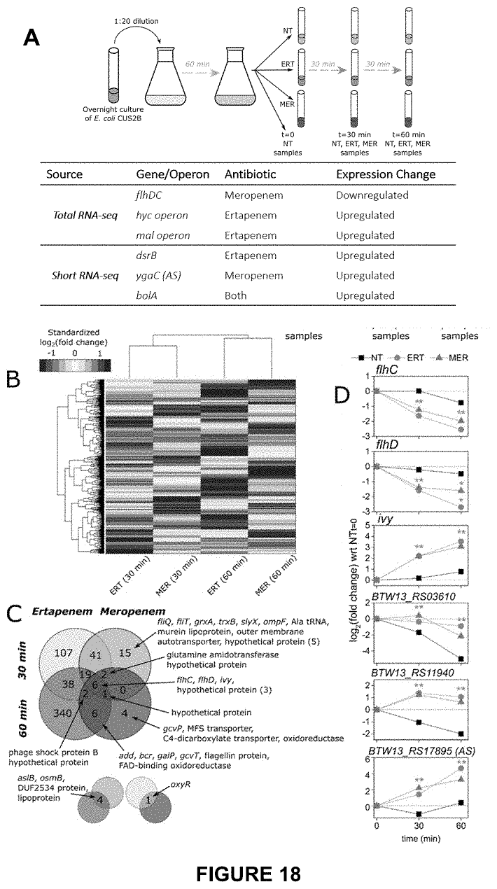

The present inventors have established the ability of PNA and the FAST platform to design effective antibiotic potentiators through the use of genomic data, using either knockouts of essential genes or resistance genes. Here, FAST PNA allow us to explore transcriptomic analysis and determine whether it can be similarly useful in reversing a bacterial resistance_phenotype. First, to reveal possible non-genetic contributions to the strain's carbapenem_resistance profile, we exposed exponentially growing E. Coli CUS2B to ertapenem and_meropenem and examined gene expression profiles after thirty and sixty minutes of treatment (FIG. 18A). This exposure time was selected based on observations from others that 30-60 minutes_is favorable for locating gene expression changes specific to antibiotic exposure. The short_time frame also greatly reduces the likelihood that the gene expression signal will be confounded by the emergence of one or more mutant genotypes. E. Coli CUS2B was diluted 1:20 from overnight cultures and grown for 1 hour to exponential phase prior to treatment with 2 μg/mL of ertapenem or 1 μg/mL of meropenem. Each carbapenem concentration is half of that required to eradicate E. Coli CUS2B under these culture conditions (conditions differ from FIG. 17 MIC)

The present inventors identified differentially expressed (DE) genes by comparing the RNA sequencing data from ertapenem- and meropenem-treated samples to an untreated control at the same timepoint. The DESeq R package was used to evaluate significance and correct P-values for multiple hypothesis testing. General expression trends were evaluated using hierarchical clustering across genes and conditions (FIG. 18B). Conditions were found to cluster by timepoint rather than antibiotic—an effect previously observed in a transcriptomic study of vancomycin-challenged Staphylococcus aureus—which suggests a generalized and transient antibiotic response. In our analysis, we detected 41 transcripts that were DE in both treatments after 30 minutes of exposure, six transcripts DE in both antibiotics after 60 minutes of exposure, and six that were DE in both treatments at 30 and 60 minutes (FIG. 18C). The six DE genes common to all conditions are indicated in FIG. 18D. Two of these genes, flhC and flhD, code for components of the transcriptional regulator FlhDC, which is responsible for regulating motility-associated functions such as swarming and flagellum biosynthesis. Both flhC and flhD genes were significantly underexpressed at 30 and 60 minutes. Perhaps relatedly, motility-associated gene ontology terms (GO:0040011: locomotion; GO:0071918: bacterial-type flagellum dependent swarming motility) were significantly overrepresented within the 30-minute overlapping set, accounting for 26 of the total 41 genes. All 26 were underexpressed. This effect was diminished by 60 minutes, with only flhC, flhD, and fliC (the gene encoding flagellin) remaining significantly underexpressed.

The gene ivy, an inhibitor of bactericidal vertebrate lysozymes, was overexpressed in both treatments at 30 and 60 minutes. Both lysozymes and carbapenems disrupt peptidoglycan polymerization, although ivy is not known to interact with these antibiotics. Three other transcripts of unknown function were overexpressed in all conditions: BTW13_RS03610 (ymgD superfamily), BTW13_RS11940 (DUF1176 superfamily), and a transcript antisense to BTW13_RS17895 (putative lipoprotein, DUF1615 superfamily). In the ertapenem response we find many more DE genes than in the meropenem response, including 38 DE genes shared between the two time points (compared with none shared across both meropenem time points). Within this set, we observed significant overrepresentation of genes related to maltodextrin transport (mal operon, GO:0042956) and the ferredoxin hydrogenase complex (hyc operon, GO:0009375). All of these overrepresented genes were found to be overexpressed in ertapenem treatments. Only three genes were underexpressed in ertapenem at both 30 and 60 minutes: 1ptG, a member of the lipopolysaccharide transport system, phoH, an ATP-binding protein, and cstA, a starvation induced peptide transporter. Of the genes specific to the meropenem response, only the flagellar biosynthesis proteins fliQ and fliT are related, and the downregulation of these genes did not continue to the 60-minute timepoint.

The present inventors also searched for differential expression in outer membrane porin operon (omp) genes, previously linked to carbapenem-resistance, and resistance-related genes identified by ARG-ANNOT. Of the omp operon, only ompF was found to be significantly DE in any condition with respect to no treatment (underexpressed in meropenem, 30 minutes), while ompA and ompC expression tracked closely with the no treatment conditions in all experiments. When expression levels of the ertapenem and meropenem experiments were directly compared at each time point, none of the three genes were found to be significantly DE. No resistance-related genes were DE in any condition.

Based on these observations, we chose three genes to target using PNA: hycA, malT, and flhC. The former two genes were chosen to probe the hyc and mal operons for their importance to resistance and their utility as antibiotic re-sensitization targets. The gene flhC was chosen to validate the consistent downregulation of the FlhDC system and evaluate whether further knockout of the gene would confer greater carbapenem resistance.

### Example 12: Differential Expression of Small RNA

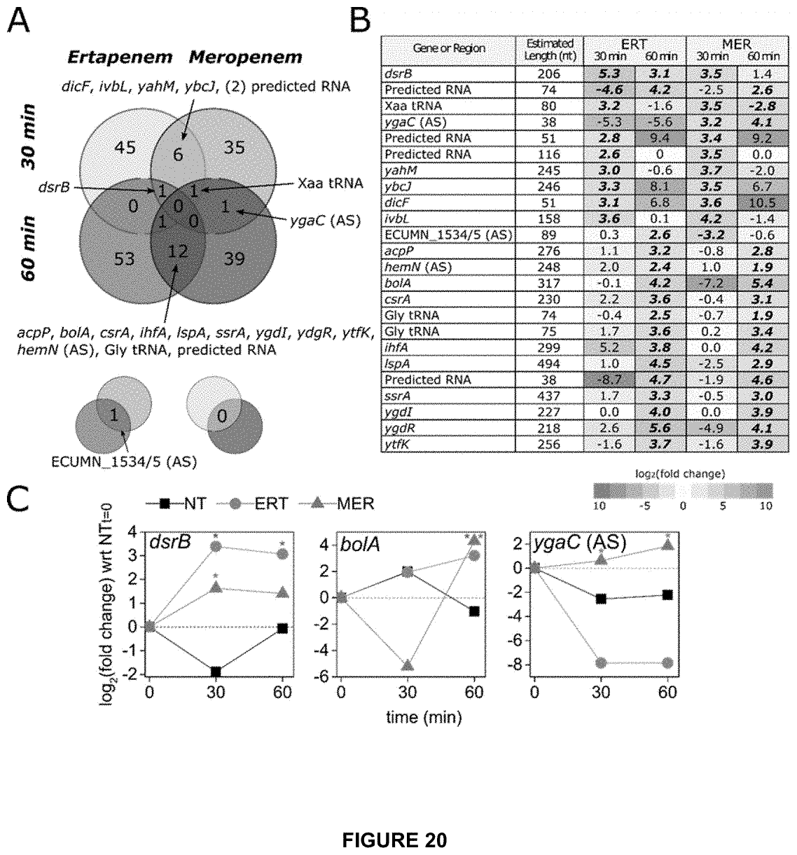

In addition to total RNA sequencing, we performed small RNA sequencing to search for resistance contributions and potential FAST PNA targets among short nucleic acids potentially involved in gene regulation. Small RNA have been previously shown to influence bacterial stress and antibiotic response. We used an RNA isolation protocol that enriched for sRNA (see_Methods) prior to sequencing, and sequencing data were aligned to the E. Coli UMN026 genome_ (the reference that maximized alignment homology) using the Rockhopper pipeline, which allowed for identification of previously documented sRNA and novel RNAs._We observe more overlap of DE genes between single time points than between_respective antibiotic treatments, suggesting a generalized and transient response similar to that_of total RNA expression (FIG. 20A). We find 22 sRNAs to be DE in at least two out of the four_conditions (FIG. 20B), including known regulatory sRNAs (dicF, ssrA), annotated short protein_coding genes (ilvB, acpP, bolA, csrA, ihfA, lspA), small putative protein-coding genes (dsrB,_yahM, ybcJ, ygdl, ygdR, ytfK), small transcripts antisense to coding genes (ygaC, hemN,_ECUN_1534/5), and novel predicted transcripts._From these lists we chose three genes to investigate with FAST PNA: bolA, dsrB, and_ygaC. bolA was chosen based on its relation to PBPs, AmpC, and the cellular stress response,_whereas the latter two were chosen to discriminate between the two carbapenem responses. The_time course of gene expression for three small transcripts of interest is presented in FIG. 20C. We_found bolA to be overexpressed in meropenem and ertapenem at 60 minutes, which may indicate_that both antibiotics are being detected and able to activate bolA, but the subsequent response is_only effective against ertapenem. dsrB was overexpressed in ertapenem at both time points, but_in meropenem only at 30 minutes. Although the function of dsrB is unknown, it is controlled by GS, the general stress response and stationary-phase sigma factor. The transcript antisense to ygaCwas overexpressed in meropenem at both timepoints but was not detected in ertapenem-treated populations. The function of ygaC is unknown, but it is controlled by the Fur transcriptional dual regulator, which suggests that it may have a role in stress response.

### Example 13: PNA Antisense Inhibition of RNA Sequencing Targets

The FAST platform comprises Design, Build, and Test modules for the creation of antisense PNA (FIG. 21A). In this study, we began our design process using transcriptomic data to generate a list of target genes, which, together with a whole-genome assembly and genome annotation, were used as inputs for the FAST tool PNA Finder. This tool was used to design multiple antisense PNA candidates for each gene target, with 12-mer sequences—a length that seeks to optimize both specificity and transmembrane transport—that were complementary to mRNA nucleotide sequences surrounding the translation start codon. PNA Finder then filtered this set of candidates to minimize the number of predicted off-targets within the E. Coli CUS2B genome, to maximize solubility, and to avoid any self-complementing sequences. For the FAST Build module, a single PNA for each gene target was selected and synthesized using Fmoc chemistry, with the cell-penetrating peptide (KFF)₃K attached at the N-terminus to improve transport across the bacterial membrane. These PNA were then tested in E. Coli CUS2B cultures in combination with each carbapenem to determine whether the two treatments would interact as predicted. A two-way ANOVA test was used to assess interaction significance, and normalized S-values (see Methods) were used to compare the observed growth to the expected growth, as predicted by the Bliss Independence Model for drug combinations.

Based on the results of our transcriptomic analysis, we selected three 235 genes identified by our total RNA sequencing analysis (hycA, malT, and flhC) and three genes identified by our small RNA sequencing analysis (bolA, dsrB, and ygaC) to be targeted by FAST PNA. Differential expression of flhC and bolA was observed in both carbapenems, while differential expression of hycA, dsrB, and the operon controlled by malT was prevalent in the ertapenem response. The transcript antisense to ygaC was overexpressed in both meropenem conditions, but the gene itself was not differentially expressed. While we suspect that the ygaC antisense transcript regulates the gene ygaC, PNA have not been established to interfere with ncRNA regulation. With this in mind, we designed a PNA to bind to the ygaC sense transcript and inhibit protein translation, to test the hypothesis that ygaC inhibition may confer greater meropenem resistance in a similar manner to the flhC-targeted PNA. The genome assembly for the clinical isolate was used by FAST to design multiple PNA for each selected gene, which were then screened for high solubility, minimal self-complementarity, and zero off-target gene inhibition in E. Coli CUS2B. Additionally, a scrambled-sequence nonsense PNA was designed to control for any possible effects of the PNA or CPP independent of sequence. No effects were found with nonsense PNA alone or in any nonsense PNA-antibiotic combination treatment. We also designed a PNA to inhibit the translation of the chromosomal β-lactamase AmpC, based on prior research showing that the enzyme can elevate ertapenem MICs. This PNA was also synthesized to assess the relative effectiveness of PNA targets selected using transcriptomic analysis, in comparison to those selected on a genomic basis.

We have previously shown the ability to re-sensitize MDR bacteria by targeting β-lactamases. In a combination of α-ampC (10 μM) in combination with ertapenem (1 μg/mL), we also observe significant synergistic interaction, evidenced both by the growth curve endpoint and cell viability assay (Table 1). E. Coli CUS2B cultures treated with a combination of 0.1 μg/mL meropenem with 10 μM α-ampC grew similarly to cultures treated with meropenem alone, as expected. Although the comparison of treatments in the cell viability assay did demonstrate significant interaction, the data does not seem to indicate a combinatorial effect, as the fluorescence level is virtually unchanged across the three treated conditions. Additionally, increasing the concentration of α-ampC to 15 μM could not resolve any effect, as the PNA alone was virtually lethal at this concentration. To determine whether transcriptomic analysis could be used by FAST to produce similar re-sensitization effects, E. Coli CUS2B was treated with each of the six PNA at a concentration of 10 μM in combination with sub-MIC carbapenem treatments (1 μg/mL ertapenem, 0.1 μg/mL meropenem). We analyzed the cultures' endpoint optical densities and cell viabilities (via resazurin assay) to assess interaction between the two treatments, based on a comparison with each individual PNA and carbapenem treatment. At these concentrations of PNA and antibiotic, we observed significant synergy between the PNAs α-hycA, α-dsrB, and α-bolA and ertapenem, with S-values of 0.23, 0.85 and 0.83 for their respective endpoint optical densities (Table 1, FIG. 20B-D). With each of these three PNAs, we performed additional interaction experiments at a PNA concentration of 15 μM with meropenem treatment to determine whether increased inhibition of these genes would demonstrate significant interaction. Of these combinations, 15 μM α-bolA demonstrated significant synergistic interaction (S=0.61) with meropenem (Table 1, FIG. 20D).

The PNA α-ygaC, α-malT, and α-flhC at concentrations of 10 μM did not exhibit significant interaction with ertapenem at 1 μg/mL or meropenem at 0.1 μg/mL. As noted above, we hypothesized that a combination of the PNA α-flhC or α-ygaC with carbapenem treatment would result in a recovery of growth and increased resistance. However, in the PNA-carbapenem combination treatments we did not observe growth recovery relative to the carbapenem-only treatment for the sub-MIC concentrations. We hypothesized that effects at such concentrations could be difficult to resolve, given that the growth curves of carbapenem treated E. Coli CUS2B reached endpoints similar to the untreated condition.

To examine this hypothesis, we treated the clinical isolate with α-flhC or α-ygaC at 10 μM in combination with ertapenem or meropenem at their MICs (FIG. 1B; 2 μg/mL and 0.25 μg/mL, respectively). At these higher carbapenem concentrations, we observed significant antagonistic interaction between both PNA and meropenem in combination (S=−0.9 and S=−0.93, respectively, for endpoint optical density) (Table 1, FIG. 20E), whereas a combination of either PNA with ertapenem showed no growth rescue (Table 1).

### Example 14: Materials and Methods

### Strain and Culture Conditions

E. Coli CUS2B was provided the Dr. Nancy Madinger, at the University of Colorado Hospital Clinical Microbiology Laboratory's organism bank. The isolate was obtained via rectal swab from a 29-year old pregnant female patient. Unless otherwise mentioned, CUS2B was propagated in aerobic conditions at 37° C. in liquid cultures of cation-adjusted Mueller Hinton broth (CAMHB) with shaking at 225 rpm or on solid CAMHB with 15 g/L of agar. Minimum inhibitory concentration assays Three colonies were picked from a plate and used to inoculate three separate overnight cultures in 1 mL CAMHB each. After 16 hours, the cultures were diluted 1:10,000 and treated with a range of antibiotic concentrations, decreasing in 2-fold increments, in a 384 or 96-well microplate using three biological replicates per condition. Growth in the plate was monitored with a Tecan GENios (Tecan Group Ltd.) running Magellan™ software (v 7.2) at an absorbance of 461 590 nm every 20 minutes for 16 hours, with shaking between measurements. The minimum inhibitory concentration was identified as the lowest antibiotic concentration preventing growth.

### Genome Sequencing

Five colonies were picked from a plate and resuspended in liquid culture. After 16 hours, 1 mL of culture was used for genomic DNA isolation with the Wizard DNA Purification Kit (Promega). Approximately 2 μg of DNA was used to prepare a paired-end 250-bp sequencing library with the Nextera XT DNA library kit. The library was sequenced on an Illumina MiSeq, resulting in 407,910 reads (20× coverage). The de novo assembly is 5,325,941 bp in length with a GC content of 50.59%. The largest contig is 394,969 bp, and the N50 is 100,215. The genome contains 5,360 protein coding sequences, 114 RNA coding sequences, 82 tRNAs, 11 ncRNAs, 260 pseudogenes, and 2 CRISPR arrays. The FASTQ files were filtered for quality using Trimmomatic (v0.32), in sliding window 473 mode with a window size of 4 bases, a minimum average window quality of 15 (phred 33 quality aligned to various E. Coli RefSeq reference genomes using Bowtie 2 (v2.2.3). SAMTools (v0.1.19) was used to remove PCR duplicates and create indexed, sorted BAM files. Variants were called and filtered using the Genome Analysis Toolkit (v2.4-9). To pass the filter, a SNP had to meet the following criteria: QD<2.0, FS >60, MQ<40.0, ReadPosRankSum <−2.0, and MappingQualityRankSum <−12.5. Filter criteria for indels was: QD <2.0, FS >60.0, and ReadPosRankSum <−2.0. A custom Python script was used to annotate variant call files using the corresponding GFF from RefSeq. For de novo assembly, reads were assembled using SPAdes (v 3.5.0) and annotation was performed with the NCBI Prokaryotic Genome Annotation Pipeline (v 4.0). Quality of the assembly was assessed using QUAST90. The FASTA generated by SPAdes was used for MLST, identification of resistance genes with ARG-ANNOT, locating CRISRRs with CRISPRfinder, and locating plasmids with PlasmidFinder.

### RNA-Sequencing and Differential Expression Analysis

Colonies were picked from a plate and resuspended in liquid culture. After 16 hours of growth, the culture was diluted 1:20 into duplicate 15 mL cultures. These were grown for 1 hour, then 3 mL from each was preserved in 2 volumes of RNAprotect. Each culture was divided into three equal parts. No antibiotic was added to one part and antibiotic were added to the other two for final concentrations of 2 μg/mL of ertapenem or 1 μg/mL of meropenem, corresponding to 50% of the MIC under these growth conditions (note that these conditions are different than the procedures used to determine the MICs in FIG. 17). After thirty and sixty minutes of growth, 1.5 mL of culture was collected from each and stored in RNAprotect. Cultures were flash frozen in ethanol and dry ice and stored at −80° C. until the time of RNA extraction. To extract RNA, samples were thawed and resuspended in 100 μL TE buffer with 0.4 mg/mL lysozyme and proteinase K. After incubation at room temperature for 5 minutes, 300 μL of lysis buffer with 20 μL/mL β-mercaptoethanol was added to each and vortexed to mix. Each lysis solution was split in half, with one half being processed for total RNA isolation and the other kit (Thermo Scientific) followed by DNase treatment with the TURBO DNA-free kit (Ambion). Small RNA was isolated using the mirVana miRNA isolation kit (Thermo Scientific). Concentration and A260/A280 were measured on a Nanodrop 2000 (Thermo Scientific). A minimum of 130 ng of RNA per sample was submitted for sequencing library preparation. Quality was further assessed with a Bioanalyzer (Agilent). Libraries were prepared using the RNAtag-Seq protocol, wherein individual samples are barcoded and pooled prior to ribosomal RNA treatment and cDNA synthesis. Here, the total RNA samples were combined into one pool, and the small RNA enriched samples were combined into a separate pool. The total RNA pool was fragmented via incubation with FastAP buffer at 94° C. After another DNAse treatment, barcoded adapters were ligated with T4 DNA ligase, then the samples were pooled and subjected to Ribo-Zero treatment. The prep for small RNA libraries was similar, without the fragmentation or ribosomal RNA treatment steps. Total RNA libraries were sequenced on a NextSeq 500 (Illumina) using a high output cycle 75-cycle run. Small RNA libraries were sequenced on a NextSeq with a medium output run, halted after 75 cycles. FASTQ files were demultiplexed with the barcode splitter function from the FastX toolbox (hannonlab.cshl.edu/fastx toolkit/, v0.0.13.2). The first seven base calls (containing the barcode) were trimmed using FastX, then adapters were removed and all reads were trimmed for quality using the sliding window mode in Trimmomatic (v0.32). Quality of the resulting FASTQ files was validated with FastQC (bioinformatics.babraham.ac.uk/projects/fastqc/). For total RNA data (including sense and antisense transcripts) and differential expression analysis, reads were aligned to the draft assembly of the CUS2B genome (available as RefSeq GCF_001910475.1) with Bowtie 2 (v2.2.3). An average of 16.5±3.2 million reads were successfully mapped per sample. SAMTools (v0.1.19) was used to create bam files. HTSeq (v0.6.1) was used to build count tables for sense and antisense reads. DESeq was used to determine differentially expressed genes, with a pooled dispersion metric and a parametric fit. Hochberg adjusted P-value was less than 0.05. For small RNA data, trimmed FASTQ files were submitted to Rockhopper which mapped to the E. Coli UMN026 genome (90,152±25,642 reads mapped per sample) and performed differential expression analysis. Small RNA transcripts were considered significantly DE if the Benjamini-Hochberg adjusted P-value was less than 0.05.

### PNA Design

PNA design was carried out using the PNA Finder toolbox. The toolbox is built using Python 2.7, the alignment program Bowtie 2, the read alignment processing program SAMtools, and the feature analysis program BEDTools. The toolbox takes a user-provided list of gene IDs and cross-references the IDs against a genome annotation file to determine the feature coordinates for each ID. The toolbox then uses these coordinates to extract PNA target sequences of a user-specified length (12 bases in this study) and user-specified positions relative to the start codon from a genome assembly FASTA file. PNA Finder provides a list of PNA candidates (the reverse complements of the target sequences) and sequence warnings regarding solubility and self-complementation. Finally, PNA Finder screens the list of PNA candidates against a user-provided genome assembly (in this study, the genome for E. Coli CUS2B) to search for off-targets and uses this analysis to filter the candidates into a final PNA list for synthesis.

### PNA Synthesis

PNA were synthesized using an Apex 396 peptide synthesizer (AAPPTec, LLC) with solid-phase Fmoc chemistry at a 10 μmol scale on MBHA rink amide resin. Fmoc-PNA monomers were obtained from PolyOrg Inc., with A, C, and G monomers protected with Bhoc groups. PNA were synthesized with the N-terminal cell-penetrating peptide (KFF) K. Cell-penetrating peptide Fmoc monomers were obtained from AAPPTec, LLC, and lysine monomers were protected with Boc groups. PNA products were precipitated in diethyl ether and purified as trifluoroacetic acid salts via reverse-phase HPLC using a C18 column.

### PNA-Antibiotic Interaction Assays

Three colonies were picked from a plate and used to inoculate three separate overnight cultures in 1 mL CAMHB each. After 16 hours, the culture was diluted 1:10,000 in a 384-well microplate using three biological replicates per condition. The total culture volume for each treatment was 50 μL. PNA were stored at −20° C. dissolved in 5% v/v DMSO in water. Growth in the plate was monitored with a Tecan GENios (Tecan Group Ltd.) running Magellan™ software (v 7.2) at an absorbance of 590 nm every 20 minutes for 24 hours, with shaking between measurements. Interaction effects were evaluated for significance using a two-way ANOVA test, and S values were calculated with respect for the expected growth inhibition as calculated by the Bliss Independence model. The S-value for a given timepoint was calculated as follows:

$$S = ( \frac{{OD}_{AB}}{{OD}_{0}} ) ( \frac{{OD}_{PNA}}{{OD}_{0}} ) - ( \frac{{OD}_{AB , PNA}}{{OD}_{0}} )$$

For a given timepoint (24 hours was used in our analyses), the variable ODAB represents optical density with only carbapenem treatment, OD0 represents the optical density without treatment, ODPNA represents optical density with only antisense-PNA treatment, and ODAB, PNA represents the optical density with a combination treatment. Plus/minus for S-values (Table 1) was calculated by propagating standard error values for each term in Equation 1. Other software and resources utilized The clustergram function from MATLAB's Bioinformatics toolbox was used for building heatmaps and dendograms. A Euclidean distance metric, optimal leaf ordering, and average linkage function were used for clustering. Ecocyc was used to gain gene names and descriptions and to define functional classes. NCBI's BLAST was used to predict gene function and to determine similarity of sequences in CUS2B to other bacterial strains. PANTHER was used for statistical overrepresentation tests, with a Bonferroni correction applied to all reported P574 values.

### Data Availability.

The whole genome shotgun sequencing data has been deposited at DDBRENA/GenBank under the accession MSDR00000000.1. The version described in this invention is version MSDR01000000. The RNA sequencing data has been deposited in NCBI's Sequence Read Archive under the accession SRP101716.

### TABLES

### REFERENCES

(1) Levy, S. B.; Marshall, B. Antibacterial Resistance Worldwide: Causes, Challenges and Responses. Nat. Med. 2004, 10 (12s), S122-S129.
(2) O'Neill, J. Antimicrobial Resistance: Tackling a Crisis for the Health and Wealth of Nations; 2014.
(3) Antibiotic/Antimicrobial Resistance www.cdc.gov/drugresistance/index.html (accessed Oct. 1, 2017).
(4) Yates, C. M.; Shaw, D. J.; Roe, A. J.; Woolhouse, M. E. J.; Amyes, S. G. B. Enhancement of Bacterial Competitive Fitness by Apramycin Resistance Plasmids from Non-Pathogenic Escherichia Coli. Biol. Lett. 2006, 2 (3), 463-465.
(5) Hoban, D. J. Antibiotics and Collateral Damage. Clin. Cornerstone 2003, 5 (SUPPL. 3).
(6) de Man, P.; Verhoeven, B. A.; Verbrugh, H. A.; Vos, M. C.; van den Anker, J. N. An Antibiotic Policy to Prevent Emergence of Resistant Bacilli. Lancet (London, England) 2000, 355 (9208), 973-978.
(7) May, A. K.; Fleming, S. B.; Carpenter, R. O.; Diaz, J. J.; Guillamondegui, O. D.; Deppen, S. A.; Miller, R. S.; Talbot, T. R.; Morris, J. A. Influence of Broad-Spectrum Antibiotic Prophylaxis on Intracranial Pressure Monitor Infections and Subsequent Infectious Complications in Head-Injured Patients. Surg. Infect. (Larchmt). 2006, 7 (5), 409-417.
(8) Antibacterial Agents in Clinical Development: An Analysis of the Antibacterial Clinical Development Pipeline, Including Tuberculosis; 2017.
(9) Hegarty, J. P.; Stewart, D. B. Advances in Therapeutic Bacterial Antisense Biotechnology. Appl. Microbiol. Biotechnol. 2017, 1-11. (10) Sully, E. K.; Geller, B. L. Antisense Antimicrobial Therapeutics. Curr. Opin. Microbiol. 2016, 33, 47-55.
(11) Giesen, U.; Kleider, W.; Berding, C.; Geiger, A.; Orum, H.; Nielsen, P. E. A Formula for Thermal Stability (T(m)) Prediction of PNA/DNA Duplexes. Nucleic Acids Res. 1998, 26 (21), 5004-5006.
(12) Demidov, V. V.; Potaman, V. N.; Frank-Kamenetskii, M. D.; Egholm, M.; Buchard, O.; Sonnichsen, S. H.; Nielsen, P. E. Stability of Peptide Nucleic Acids in Human Serum and Cellular Extracts. 1994, 48 (6), 1310-1313.
(13) Nielsen, P. E.; Egholm, M.; Berg, R. H.; Buchardt, O. Sequence-Selective Recognition of DNA by Strand Displacement with a Thymine-Substituted Polyamide. Science (80-.). 1991, 254 (5037), 1497 LP −1500.
(14) Choi, J. J.; Jang, M.; Kim, J.; Park, H. Highly Sensitive PNA Array Platform Technology for Single Nucleotide Mismatch Discrimination. J. Microbiol. Biotechnol. 2010, 20 (2), 287-293.
(15) Good, L.; Awasthi, S. K.; Dryselius, R.; Larsson, O.; Nielsen, P. E. Bactericidal Antisense Effects of Peptide-PNA Conjugates. Nat. Biotechnol. 2001, 19 (4), 360-364.
(16) Mondhe, M.; Chessher, A.; Goh, S.; Good, L.; Stach, J. E. M. Species-Selective Killing of Bacteria by Antimicrobial Peptide-PNAs. PLoS One 2014, 9 (2).
(17) Good, L.; Nielsen, P. E. Inhibition of Translation and Bacterial Growth by Peptide Nucleic Acid Targeted to Ribosomal RNA. Biochemistry 1998, 95 (March), 2073-2076.
(18) Soofi, M. A.; Seleem, M. N. Targeting Essential Genes in Salmonella Enterica Serovar Typhimurium with Antisense Peptide Nucleic Acid. 2012, 56 (12), 6407-6409.
(19) Kurupati, P.; Tan, K. S. W.; Kumarasinghe, G.; Poh, C. L. Inhibition of Gene Expression and Growth by Antisense Peptide Nucleic Acids in a Multiresistant β-Lactamase-Producing Klebsiella Pneumoniae Strain. Antimicrob. Agents Chemother. 2007, 51 (3), 805-811.
(20) Otsuka, T.; Brauer, A. L.; Kirkham, C.; Sully, E. K.; Pettigrew, M. M.; Kong, Y.; Geller, B. L.; Murphy, T. F. Antimicrobial Activity of Antisense Peptide-Peptide Nucleic Acid Conjugates against Non-Typeable Haemophilus Influenzae in Planktonic and Biofilm Forms. J. Antimicrob. Chemother. 2017, 72 (1), 137-144. (21) Bai, H.; You, Y.; Yan, H.; Meng, J.; Xue, X.; Hou, Z.; Zhou, Y.; Ma, X.; Sang, G.;
Luo, X. Antisense Inhibition of Gene Expression and Growth in Gram-Negative Bacteria by Cell-Penetrating Peptide Conjugates of Peptide Nucleic Acids Targeted to RpoD Gene. Biomaterials 2012, 33 (2), 659-667.
(22) Courtney, C. M.; Chatterjee, A. Sequence-Specific Peptide Nucleic Acid-Based Antisense Inhibitors of TEM-1 Beta-Lactamase and Mechanism of Adaptive Resistance. ACS Infect. Dis. 2016, 1 (6), 253-263.
(23) Langmead, B.; Salzberg, S. L. Fast Gapped-Read Alignment with Bowtie 2. Nat. Methods 2012, 9, 357.
(24) Li, H.; Handsaker, B.; Wysoker, A.; Fennell, T.; Ruan, J.; Homer, N.; Marth, G.; Abecasis, G.; Durbin, R. The Sequence Alignment/Map Format and SAMtools. Bioinformatics 2009, 25 (16), 2078-2079.
(25) Quinlan, A. R.; Hall, I. M. BEDTools: A Flexible Suite of Utilities for Comparing Genomic Features. 2017, 26 (6), 841-842.
(26) Szklarczyk, D.; Gable, A. L.; Lyon, D.; Junge, A.; Wyder, S.; Huerta-Cepas, J.; Simonovic, M.; Doncheva, N. T.; Morris, J. H.; Bork, P.; et al. STRING V11: Protein-Protein Association Networks with Increased Coverage, Supporting Functional Discovery in Genome-Wide Experimental Datasets. Nucleic Acids Res. 2019, 47 (D1), D607-D613.
(27) Dryselius, R.; Aswasti, S. K.; Rajarao, G. K.; Nielsen, P. E.; Good, L. The Translation Start Codon Region Is Sensitive to Antisense PNA Inhibition in Escherichia Coli. Oligonucleotides 2003, 13 (6), 427-433.
(28) Gildea, B. D.; Coull, J. M. Methods for Modulating the Solubility of Synthetic Polymers. U.S. Pat. No. 6,770,442 B2, 2004.
(29) Doyle, D. F.; Braasch, D. A.; Simmons, C. G.; Janowski, B. A.; Corey, D. R. Inhibition of Gene Expression inside Cells by Peptide Nucleic Acids: Effect of MRNA Target Sequence, Mismatched Bases, and PNA Length. Biochemistry 2001, 40 (1), 53-64.
(30) Weiler, J.; Gausepohl, H.; Hauser, N.; Jensen, 0. N.; Hoheisel, J. D. Hybridisation Based DNA Screening on Peptide Nucleic Acid (PNA) Oligomer Arrays. Nucleic Acids Res. 1997, 25 (14), 2792-2799.

1. A method for the rational design and production of therapeutic oligomers comprising:
generating a genomic library for an organism from known target genes, whole or partial genome assemblies, or biosynthetic gene clusters (BGC's) derived from microbiome gene analysis;
initiating a sequence identification function comprising the steps of:
analyzing said genomic library and identifying a plurality of prospective gene targets whose expression may be regulated by a proposed therapeutic oligomer;
generating a proposed therapeutic oligomer sequence corresponding to each of said prospective gene targets;
outputting a sequence warning for any of said proposed therapeutic oligomer sequences;
initiating an off-target sequence function to identify genomic loci that said proposed therapeutic oligomer is predicted to bind comprising the steps of:
searching for incidental alignments between said proposed therapeutic oligomer sequence and said genomic library;
aligning each of said proposed therapeutic oligomer sequences to its corresponding genome assembly location and applying a user-specified number of allowed mismatches, using the proposed therapeutic oligomer sequence length parameter as the seed length;
identifying whether one or more of said proposed therapeutic oligomer sequences overlaps with any genomic features of said genomic library;
outputting a file identifying all potentially inhibitory alignments of said proposed therapeutic oligomer sequences;
outputting a file identifying all potentially off-target alignments of said proposed therapeutic oligomer sequences;
selecting one or more of said proposed therapeutic oligomer sequences wherein said selection is based on at least one of the following criteria:
inhibition of said target gene expression;
upregulation of said target gene expression;
solubility of said proposed therapeutic oligomer;
stability of said proposed therapeutic oligomer;
presence of self-complementary subsequences in said proposed therapeutic oligomer;
off-target alignments in coding sequences;
coding sequence alignments that occur near a start codon of said target gene;
synthesizing one or more of said proposed therapeutic oligomer sequences; and
testing one or more of said proposed therapeutic oligomer sequences.

2-4. (canceled)

5. The method of claim 2, wherein said therapeutic oligomer comprises peptide nucleic acid (PNA).

6. The method of claim 5, wherein said PNA inhibits gene expression in a target host or upregulates gene expression in a target host.

7. The method of claim 6, wherein said prospective gene targets comprise essential genes selected from the group consisting of: pathogenicity genes; antibiotic resistance genes; metabolism genes; radiation responsive genes; genes associated with an immune response; genes associated with a disease condition; oncogenes; anti-inflammatory genes, or a combination of the same.

8. The method of claim 5, wherein said PNA comprises a 12-mer PNA.

9-10. (canceled)

11. The method of claim 5, wherein said PNA is synthesized using solid-state PNA synthesis using Fmoc chemistry.

12. The method of claim 1, wherein said step of synthesizing comprises the step of automated and high-throughput parallel synthesizing a library of therapeutic oligomer sequences.

13-14. (canceled)

15. The method of claim 1, wherein said step of testing comprising the step of testing the efficacy or toxicity of said proposed therapeutic oligomer sequences in an in vitro or in vivo system.

16. The method of claim 15, wherein said step of testing the efficacy and/or toxicity of said proposed therapeutic oligomer sequences comprises the step of testing the efficacy or toxicity of said proposed therapeutic oligomer sequence in a macrophage based host-infection model.

17. A system for the rational design and production of therapeutic oligomers comprising:
a sequence identification function configured to identify gene targets from one or more genetic databases for a target host;
a therapeutic oligomer identification and generation function comprising a target identification function configured to identify genomic loci that a therapeutic oligomer is predicted to bind, and further configured to design a plurality of unique therapeutic oligomers that exhibit at least one of the following:
upregulate or downregulate expression of one or more gene targets in said host; and/or
reduced chance of off-target effect by comparison to different host strains, target host microbiome, and target host transcriptomes;
an automated high-throughput therapeutic oligomer production module configured to generate said unique therapeutic oligomers;
a testing module configured to evaluate the efficacy and/or toxicity of said unique therapeutic oligomers; and
a delivery system configured to deliver said unique therapeutic oligomers to a host cell.

18-20. (canceled)

21. The system of claim 18, wherein said therapeutic oligomers comprises peptide nucleic acids (PNA).

22. The system of claim 21, wherein said PNA inhibits gene expression in a host cell or upregulates gene expression in a host cell.

23. The method of claim 22, wherein said prospective gene targets comprise essential genes selected from the group consisting of: pathogenicity genes; antibiotic resistance genes; metabolism genes; radiation responsive genes; genes associated with an immune response; genes associated with a disease condition; oncogenes; anti-inflammatory genes, or a combination of the same.

24. The system of claim 21, wherein said PNA comprises a 12-mer PNA.

25-26. (canceled)

27. The system of claim 21, wherein said PNA is synthesized using solid-state PNA synthesis using Fmoc chemistry.

28. The system of claim 17, wherein said automated high-throughput therapeutic oligomer production module comprises a parallel automated high-throughput therapeutic oligomer production module configured to produce a library of therapeutic oligomer sequences.

29-30. (canceled)

31. The system of claim 17, wherein said testing module comprises a testing module configured to evaluate the efficacy or toxicity of said unique therapeutic oligomers in an in vitro or in vivo system

32. The system of claim 31, wherein said testing module configured to evaluate the efficacy and/or toxicity of said unique therapeutic oligomers comprises testing module configured to evaluate the efficacy or toxicity of said unique therapeutic oligomers in a macrophage based host-infection model.

33. The system of claim 17, wherein said sequence identification function comprises a Get Sequence function.

34. The system of claim 17, wherein said therapeutic oligomer identification and generation function comprises a Find-Off Targets function.

35-85. (canceled)
